(19) United States
(12) Patent Application Publication
(10) Pub. No.: US 2005/0271631 A1
(43) Pub. Date: Dec. 8, 2005

(54) Material compositions and related systems and methods for treating cardiac conditions

(76) Inventors: Randall J. Lee, Hillsborough, CA (US); Karen Christman, Carpinteria, CA (US); Richard Sievers, Petaluma, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/129,046

(22) Filed: May 12, 2005

Related U.S. Application Data
(63) Continuation of application No. PCT/US03/23162, filed on Jul. 25, 2003.
(60) Provisional application No. 60/429,914, filed on Nov. 29, 2002.
(60) Provisional application No. 60/431,287, filed on Dec. 6, 2002.

(51) Int. Cl. A61K 45/00

(52) U.S. Cl. 424/93.7

(57) ABSTRACT

A medical condition associated with a cardiac structure is treated by injecting an injectable polymer agent into the cardiac structure such that a therapeutic mechanical scaffolding is formed within the cardiac structure itself. In particular, the injectable scaffolding agent is a fibrin glue agent and is injected into regions of damaged myocardium such as ischemic tissue or infarct. LV wall dysfunction may also be treated by injecting the scaffolding agent into the LV wall. Cell therapy may be combined with the injection of fibrin glue or other injectable polymer scaffold agent. The polymeric forms of the agent may be injectable as precursor materials that polymerize as a scaffold in-situ within the cardiac structure. In other modes, polymer agents are injected in order to provide therapeutic angiogenesis, or to induce deposition of cells within the injected area, such as by providing the polymer with fragment E or RDG binding sites, respectively.

### CROSS-REFERENCE TO RELATED APPLICATIONS

This application claims priority from, and is a 35 U.S.C. § 111 (a) continuation of, co-pending PCT international application serial number PCT/US2003/023162 filed on Jul. 25, 2003 which designates the US, incorporated herein by reference in its entirety, which claims priority to U.S. provisional patent application Ser. No. 60/429,914, filed on Nov. 29, 2002, incorporated herein by reference in its entirety, and also claims priority to U.S. provisional patent application Ser. No. 60/431,287, filed on Dec. 6, 2002, incorporated herein by reference in its entirety. Priority is claimed to each of the foregoing applications.

The foregoing PCT international application was published as International Publication No. WO 2004/050013 A2 on Jun. 17, 2004, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to therapeutic agents and related delivery systems and methods for treating cardiac conditions in living beings, and more particularly for treating cardiac conditions generally associated with dilated cardiomyopathy, myocardial infarctions, or congestive heart failure. Still more specifically, it is related to using injectors to deliver injectable scaffolding agents into cardiac structures so as to form therapeutic internal wall scaffoldings.

2. Description of Related Art

Cardiovascular disease (CVD) is the leading cause of death in the United States. The American Heart Association estimated 60 million patients in the United States have CVD costing the healthcare system approximately $186 billion annually. There are approximately 550,000 new cases of congestive heart failure (CHF) each year with the incidence approaching 10 per 1,000 population in those older than age 65. The 5-year mortality rate for CHF is about 50%, and in patients with CHF, sudden cardiac death occurs at a rate 6-9 times that of the general population. Coronary artery disease is the leading cause of heart failure in the United States.

Further information related to the prevalence of CVD and CHF in particular is disclosed in the following publications which are herein incorporated in their entirety by reference thereto: Lenfant, C., “Fixing the failing heart.” 1997; Circulation 95:771-772; “Heart and Stroke Statistical Update,” American Heart Association, 2001; Lenfant, C. “Cardiovascular research: an NIH perspective.” 1997; Cardiovasc. Surg. 5:4-5; Cohn, J. N., et al., “Report of the National Heart, Lung, and Blood Institute Special Emphasis Panel on Heart Failure Research.” 1997; Circulation 95:766-770.

Heart failure following a myocardial infarction (MI) is often progressive. Scar tissue formation and aneurismal thinning of the infarct region often occur in patients who survive myocardial infarctions. It is believed that the death of cardiomyocytes results in negative left ventricular (LV) remodeling which leads to increased wall stress in the remaining viable myocardium. This process results in a sequence of molecular, cellular, and physiological responses which lead to LV dilation. Although the exact mechanisms of heart failure are unknown, LV remodeling is generally considered an independent contributor to its progression.

Coronary heart disease is the leading cause of death in the United States. According to the American Heart Association an estimated 1.1 million Americans will suffer from a new or recurrent coronary attack this year. Cardiac transplantation is currently the only treatment for hearts that are severely damaged due to MI. Given the chronic shortage of donor hearts, alternate strategies are needed to improve the lives of those with heart failure. The emerging field of tissue engineering may provide promising alternatives.

Previously disclosed tissue engineering approaches for cardiac therapy are generally intended to repair lost or damaged tissue through the use of cellular transplantation and biomaterial scaffolds. Several groups have disclosed methods intended to improve cardiac function through the injection of cells alone into ischemic myocardium. One group also disclosed suturing fetal cardiomyocyte-seeded alginate gels to the epicardial surface in order to preserve LV function.

Negative left ventricular remodeling is believed to contribute independently to the progression of heart failure following a myocardial infarction. Several prior attempts have been disclosed with the intended purpose of providing mechanical external constraints as external support to limit negative left ventricular remodeling.

One previously disclosed study included suturing a polymeric mesh to the epicardial surface for the intended purpose of providing an external support to prevent LV dilation and deterioration of LV function post-MI. Another previously disclosed device that has been investigated provides a plurality of sutures that are implanted in an open-chest procedure across the ventricle under tension to provide a change in the ventricle shape and a decrease chamber diameter. This trans-cavitary suture network is intended to decrease the radius of the ventricle to thus reduce ventricular wall stress. Another previously disclosed device under clinical investigation is generally a mesh structure that is implanted as a jacket around the heart and adjusted to provide a snug fit during open-chest surgery. It is intended that the jacket restrains the heart from further enlargement. Still another approach being investigated provides a nitinol mesh as a similar external restraining device to that described above; however, the super-elastic system is intended to assist in systolic contraction, and is generally intended for use via thorascopically guided minimally invasive delivery. Still another system being investigated includes a rigid ring that is implanted during open-chest surgery as another external constraining device to the ventricle. This ring is intended to decrease ventricular wall stress and prevent further enlargement of the heart by reducing the radius and modifying the shape of the ventricle. Yet another device approach that was at one time being investigated includes a radiofrequency (“RF”) ablation catheter intended to shrink damaged, i.e. infarcted scar, tissue during cardiac surgery.

Additional examples of devices and methods similar to one or more of those discussed above have been disclosed by various companies, including the following: “Acorn;” “Myocor;” “Paracor;” “Cardioclasp;” and “Hearten.”

Still further more detailed examples of cardiac tissue conditions, devices and systems intended to provide interventional solutions for various medical conditions, tissue engineering materials and techniques, research tools, and various tissue culturing and intended cellular therapy methods, are variously disclosed in the following references for further background understanding:
1. Taylor D A, et al. Regenerating functional myocardium: improved performance after skeletal myoblast transplantation. Nat Med. 1998; 4:929-33.
2. Leor J, et al. “Bioengineered cardiac grafts: A new approach to repair the infarcted myocardium?” Circulation. 2000; 102:III56-61.
3. Cleutjens J P, et al., “Regulation of collagen degradation in the rat myocardium after infarction.” J Mol Cell Cardiol. 1995; 27:1281-92.
4. Erlebacher J A, et al., “Early dilation of the infarcted segment in acute transmural myocardial infarction: role of infarct expansion in acute left ventricular enlargement.” J Am Coll Cardiol. 1984; 4:201-8.
5. Olivetti G, et al., “Side-to-side slippage of myocytes participates in ventricular wall remodeling acutely after myocardial infarction in rats.” Circ Res. 1990; 67:23-34.
6. Pfeffer M A, et al., “Ventricular remodeling after myocardial infarction. Experimental observations and clinical implications.” Circulation. 1990; 81:1161-72.
7. Warren S E, et al., “Time course of left ventricular dilation after myocardial infarction: influence of infarct-related artery and success of coronary thrombolysis.” J Am Coll Cardiol. 1988; 11:12-9.
8. Hunyadi J, et al., “Keratinocyte grafting: a new means of transplantation for full-thickness wounds.” J Dermatol Surg Oncol. 1988; 14:75-8.
9. Horch R E, et al., “Single-cell suspensions of cultured human keratinocytes in fibrin-glue reconstitute the epidermis.” Cell Transplant. 1998; 7:309-17.
10. Andree C, et al., “Plasmid gene delivery to human keratinocytes through a fibrin-mediated transfection system.” Tissue Eng. 2001; 7:757-66.
11. Sims C D, et al., “Tissue engineered neocartilage using plasma derived polymer substrates and chondrocytes,” Plast Reconstr Surg. 1998; 101:1580-5.
12. Bach A D, et al., “Fibrin glue as matrix for cultured autologous urothelial cells in urethral reconstruction.” Tissue Eng. 2001; 7:45-53.
13. Han B, et al., “A fibrin-based bioengineered ocular surface with human corneal epithelial stem cells.” Cornea. 2002; 21:505-10.
14. Watanabe E, et al., “Cardiomyocyte transplantation in a porcine myocardial infarction model.” Cell Transplant. 1998; 7:239-46.
15. Chawla P S, et al., “Angiogenesis for the treatment of vascular diseases.” Int Angiol. 1999; 18:185-92.
16. Kipshidze N, et al. “Angiogenesis in a patient with ischemic limb induced by intramuscular injection of vascular endothelial growth factor and fibrin platform.” Tex Heart Inst J. 2000; 27:196-200.
17. Sakiyama-Elbert S E, Hubbell J A. “Development of fibrin derivatives for controlled release of heparin-binding growth factors.” J Control Release. 2000; 65:389-402.
18. Pandit A S, Feldman D S, Caulfield J, et al. “Stimulation of angiogenesis by FGF-1 delivered through a modified fibrin scaffold.” Growth Factors, 1998; 15:113-23.

The disclosures of each of the references provided immediately above, or as elsewhere indicated in this disclosure, are herein incorporated in their entirety by reference thereto.

The disclosures of the following issued U.S. patents are also herein incorporated in their entirety by reference thereto: U.S. Pat. No. 5,103,821 to King; U.S. Pat. No. 6,151,525 to Soykan et al.; and U.S. Pat. No. 6,238,429 to Markowitz et al. The disclosures of the following PCT International Patent Application Publications are also herein incorporated in their entirety by reference thereto: WO 90/10471 to King; and WO 98/02150 to Stokes et al.

There is a need for providing a wall support or tissue engineering scaffold within cardiac structures themselves.

There is a need for therapeutic, injectable scaffolding agents and related systems and methods adapted to inject such agents into cardiac structures as an internal wall scaffold and/or tissue engineering scaffold.

There is a need for improved materials and related systems and methods for treat ischemic myocardium, such as associated with myocardial infarction.

There is also a need for injectable solutions to provide support to damaged cardiac structures, such as infracted regions of ventricles in the heart.

There is also a need to provide support to the ventricle within the ventricular wall itself.

There is also still a need to provide angiogenesis into cardiac tissue structures receiving cell implant therapy, such as within infarcted ventricle walls.

There is also still a need to provide for additional cellular recruitment and deposition into cardiac tissue structures receiving cell implant therapy.

There is also still a need to provide a scaffold for enhanced retention and viability of implanted cells within cardiac tissue structures.

### BRIEF SUMMARY OF THE INVENTION

Accordingly, various aspects of the invention are provided as follows.

One aspect of the invention is a system and method adapted to prevent left ventricular wall dysfunction.

Another aspect of the invention is a system and method adapted to prevent negative left ventricular wall remodeling.

Another aspect of the invention is a system and method adapted to treat infarcted regions of cardiac chamber walls.

Another aspect of the invention is a system and method adapted to provide a therapeutic scaffolding within a cardiac structure of a heart in a patient.

Another aspect of the invention is a system and method adapted to enhance retention of transplanted cells in a patient.

Another aspect of the invention is a system and method adapted to provide an injectable scaffolding agent for injection into cardiac structures.

Another aspect of the invention is a system and method for injecting therapeutic, internal wall scaffolding within cardiac structures.

Another aspect of the invention is a system and method adapted to provide therapeutic mechanical scaffolding within a cardiac structure as an internal wall support.

Another aspect of the invention is a system and method adapted to induce or enhance therapeutic angiogenesis in cardiac structures or injected cardiac structure scaffolds.

Another aspect of the invention is a system and method adapted to provide therapeutic angiogenesis to transplanted cells within a patient.

Another aspect of the invention is a system and method adapted to enhance deposition of cells within a patient into a cardiac structure.

Another aspect of the invention is a system and method adapted to treat cardiac conditions following myocardial infarction.

Another aspect of the invention is a system and method adapted to treat ischemic cardiac tissue structures.

Another aspect of the invention is a system and method adapted to treat infarcts.

Another aspect of the invention is a system and method adapted to treat cardiac conditions associated with congestive heart failure.

Another aspect of the invention is a system and method adapted to treat cardiac conditions associated with cardiomyopathy.

It is to be appreciated that further more detailed aspects of the invention are also contemplated as beneficial with respect to achieving the objectives of one or more of the preceding aspects, or otherwise providing other substantial benefits as would be apparent to one of ordinary skill, including for example as follows.

The invention in one such further aspect is a preparation of material that is adapted to be implanted into a region of myocardium and to provide an internal wall support and tissue engineering scaffold to at least a portion of the heart. In one mode, the preparation is particularly adapted to be injected into the region in a manner adapted to treat the ischemic myocardium. In another mode, the material is injectable. In one highly beneficial embodiment of this mode, the material is an injectable biopolymer. In still a further highly beneficial variation of this embodiment, the injectable biopolymer is an injectable fibrin glue material.

Another aspect of the invention is a method for treating ischemic myocardium that includes implanting a material into a region of myocardium so as to provide an internal wall support and tissue engineering scaffold to at least a portion of the heart. Another aspect of the invention is a method for treating a heart of a patient that includes implanting a material into a region of myocardium in a heart of a patient so as to treat a cardiac condition associated with ischemic myocardium in the heart. One mode of this aspect includes treating the ischemic myocardium by providing an internal wall support and tissue engineering scaffold to at least a portion of the heart. Another mode of this aspect includes preventing negative remodeling of the heart with respect to the ischemic myocardium.

One further mode of these method aspects further includes injecting a material into the region. One beneficial embodiment of this mode includes injecting a biopolymer into the region. A highly beneficial variation of this embodiment includes injecting a fibrin glue into the region.

Another aspect of the invention is a system for treating a cardiac condition in a patient that includes a volume of living cells and a volume of an injectable polymer agent that are combined as an injectable scaffolding agent that is adapted to provide a therapeutic mechanical scaffolding when injected into a cardiac structure.

Another aspect of the invention is a method for treating a cardiac condition in a heart of a patient that includes injecting a volume of non-living polymer agent into a cardiac structure associated with the heart in a manner which forms a therapeutic scaffolding to the cardiac structure.

Another aspect of the invention is a system for treating a cardiac condition associated with a heart of a patient that includes a cardiac structure injector in combination with a means for providing a therapeutic scaffolding within a cardiac structure associated with the heart.

Another aspect of the invention is a system for treating a cardiac condition associated with a heart in a patient that includes a cardiac structure injector coupled to a volume of living cells such that the cardiac structure injector is adapted to inject the volume of living cells into a cardiac structure associated with the heart. This aspect further includes a means coupled to the cardiac structure injector for enhancing the retention of the living cells injected into the cardiac structure.

Another aspect of the invention is a system for treating a cardiac condition associated with a heart in a patient, and includes a volume of injectable polymer agent provided together with a means for treating the cardiac condition with the volume of injectable polymer agent.

Another aspect of the invention is a method for treating a cardiac condition associated with a heart in a patient, and includes coupling an injectable polymer agent to a cardiac structure injector in combination with the step of injecting the injectable polymer agent into a cardiac structure with the cardiac structure injector for treating a condition associated with the cardiac structure.

Another aspect of the invention is a method for treating LV wall dysfunction associated with a left ventricle of a heart in a patient, and includes injecting a volume of injectable polymer agent into the left ventricle of the heart. The injected volume of polymer agent is adapted to form at least in part a therapeutic scaffolding sufficient to treat the LV wall dysfunction.

Another aspect of the invention is a method for treating ischemia associated with a cardiac structure of a heart in a patient, and includes injecting a volume of injectable polymer agent into the ischemic cardiac structure. The injected volume of polymer agent is adapted to at least in part treat the ischemic cardiac structure.

Another aspect of the invention is a method for treating a cardiac condition associated with a heart in a patient, and includes injecting a polymer agent into a cardiac structure associated with the cardiac condition, and further includes inducing angiogenesis at least in part with the polymer agent injected into the cardiac structure.

Another aspect of the invention is a method for treating a cardiac condition associated with a heart in a patient that includes: injecting a polymer agent into a cardiac structure associated with the cardiac condition, and inducing deposition of autologous cells within the patient at least in part with the polymer agent injected into the cardiac structure.

Another aspect of the invention is a method for treating a cardiac condition in a heart of a patient, and includes injecting a volume of injectable polymer agent into a cardiac structure associated with the cardiac condition, and also injecting a volume of living cells into the cardiac structure. The injected volume of living cells and the injected volume of non-living polymer are combined to provide a therapeutic scaffolding in the cardiac structure.

Another aspect of the invention is a method for treating a cardiac condition in a heart of a patient, and includes injecting a volume of injectable polymer agent into a cardiac structure associated with the cardiac condition, and injecting a volume of living cells into the cardiac structure. The injected volume of polymer agent enhances retention of the injected living cells within the cardiac structure.

Another aspect of the invention is a method for treating an infarct region associated with a heart of a patient, and includes injecting a volume of living cells into the infarct region, and also injecting a volume of non-living polymer into the infarct region. The injected volume of living cells and the injected volume of non-living polymer are combined in the infarct region to provide a therapeutic effect to the heart.

### BRIEF DESCRIPTION OF THE SEVERAL VIEWS OF THE DRAWING(S)

FIG. 1 shows a schematic illustration of injection procedure for cells in combination with a fibrin glue agent according to certain aspects of the invention.

FIG. 2 shows a schematic view of another needle injection assembly according to certain aspects of the invention.

FIGS. 3A-C show various cross-sectioned views of certain catheter shaft arrangements corresponding with further embodiments taken along line 2-2 of FIG. 2.

FIG. 4 shows a schematic side view of one particular system arrangement for a cardiac structure injection assembly coupled to a source of injectable scaffolding agent according to further aspects of the invention.

FIG. 5A shows a schematic view of an injectable scaffolding agent system with a cross-sectioned view of one illustrative injection needle embodiment according to further aspects of the invention.

FIG. 5B shows an enlarged view of an injected drop of scaffolding agent.

FIG. 6 shows a cross-sectioned view of another needle injection assembly during one mode of use, and schematically shows the injection needles coupled to a source of injectable scaffolding agent.

FIG. 7 shows a plan view of an illustrative region of damaged tissue associated with a cardiac structure such as along a left ventricular wall.

FIG. 7B shows a schematic view of a cardiac structure delivery assembly similar to that shown in FIG. 4 during one mode of use for treating the damaged cardiac structure shown in FIG. 7A.

FIG. 7C shows a schematic plan view of a therapeutic mechanical scaffolding resulting from the mode of use embodiment shown in FIG. 7B.

FIGS. 8A-B schematically illustrate certain aspects related to interstitial cell coupling in relation to therapeutic scaffolding provided according to certain embodiments of the invention.

FIG. 9A shows a cross-sectioned view of a heart that includes an infarcted or otherwise ischemic area of the left ventricle wall prior to treatment according to the invention.

FIG. 9B shows the same view of the heart shown in FIG. 9A, except during one endocardial mode of using the invention to treat the damaged area of the left ventricle wall.

FIG. 9C shows the same view of the heart shown in FIGS. 9A-B, except during another endocardial mode of use.

FIGS. 10A-C show various views of one particular needle injection assembly according to another embodiment of the invention.

FIG. 11 shows certain further detail of another injection needle assembly according to a further embodiment.

FIG. 12 shows a cross-sectioned view of another heart with a further needle injection assembly shown during use in treating another area of damaged left ventricle wall.

FIGS. 13A-B show various views taken along lines A-A and B-B, respectively, of FIG. 12.

FIGS. 14A-B show various views of another cardiac structure delivery catheter incorporating an expandable member in conjunction with injection needles coupled to a source of injectable scaffolding agent, wherein FIG. 14B is a view taken along line B-B of FIG. 14A.

FIG. 15 shows one mode of transvascular use of a cardiac structure delivery catheter similar to one of the embodiments shown in FIGS. 15A-B.

FIGS. 16A-B shows a schematic views of further respective modes of transvascular use for a cardiac structure delivery catheter to inject scaffolding agent into a damaged area of cardiac structure such as a left ventricle wall.

FIGS. 17A-B show two further embodiments for cardiac structure delivery catheters, respectively, adapted to deliver injectable scaffolding agent to damaged cardiac structures.

FIG. 18 shows a schematic view of one particular combination system for providing cardiac treatment using injectable scaffoldings.

FIG. 19 shows a photomicrograph of hematoxylin and eosin stained fibrin glue (original magnification ×100).

FIG. 20 shows photomicrographs of hematoxylin and eosin stained left ventricular free wall transmural slices. Extensive transmural myocardial infarctions are visible in all sections. A is a section from a control heart. B is from a heart that received fibrin glue alone. C is from a heart that was injected with only myoblasts. D is a section from a heart receiving myoblasts in fibrin glue. Note the thin infarct wall of the control group section (original magnification ×10).

FIG. 21 shows reverse contrast negative image of immunostaining for the skeletal fast isoform of myosin heavy chain. Both pictures are from a section of a heart in the cells in fibrin group (A: original magnification ×100, B: original magnification ×400).

FIG. 22 shows various panels A-D of stained cross-sections of certain tissue samples prepared during the experiments conducted according to Example 2.

FIG. 23 shows various panels A-D of additional stained cross-sections of certain tissue samples also prepared during the experiments conducted according to Example 2.

FIG. 24 shows a bar graph demonstrating infarct size as determined by percent of the LV was measured for each group according to the experiment conducted under Example 2.

FIG. 25 shows a bar graph demonstrating arteriole density within infarct, at the border of infarcts, and total, for each respective group related to the experiment conducted under Example 2.

FIG. 26 shows two respective panels A-B for stained tissue cross-sections taken during the experiment according to Example 2.

FIG. 27 shows a bar graph comparing myoblast density for samples treated with cells in BSA versus cells in Fibrin according to the experiments of Example 3.

FIG. 28 shows a bar graph comparing arteriole density for samples receiving BSA injections versus samples receiving Fibrin injections according to further aspects of the experiments of Example 3.

It is to be appreciated that certain of the Figures representing pictures of stained tissue cross-sectioned have been provided in reverse contrast or otherwise contrast-modified form from their original stained view in order to allow one of ordinary skill in the art an opportunity to view the various structures in conjunction with the accompanying written description.

### DETAILED DESCRIPTION OF THE INVENTION

It will be appreciated that the apparatus may vary as to configuration and as to details of the parts, and that the method may vary as to the specific steps and sequence, without departing from the basic concepts as disclosed herein.

This invention, and in particular by reference to the various embodiments herein shown and described, is related to injecting polymer agent materials into cardiac tissue in order to treat various medical conditions, such as for example dilated cardiomyopathies, and in more specific examples conditions associated with congestive heart failure or acute myocardial infarction (such as for example treating ischemic tissue or infarcts).

Coronary artery disease and myocardial ischemia with infarction is the etiology in the majority of patients with dilated cardiomyopathies (DCM). DCM is characterized by left ventricular dilation, normal or decreased wall thickness and reduced ventricular systolic function. LV aneurysm is a type of ischemic cardiomyopathy in which a large transmural MI thins and expands over time. It has become clear that aneurysm formation begins early after myocardial infarction (MI). Further related information is disclosed in the following references: Giles, T., “Dilated Cardiomyopathy, in Heart Failure,” P. Poole-Wilson, et al., Editors, 1997, Churchill Livingstone: New York, p. 401-422; and Eaton, L. W., et al., “Regional cardiac dilatation after acute myocardial infarction: recognition by two-dimensional echocardiography,” N Engl J Med, 1979. 300(2): p. 57-62).

The myocardial infarct scar can result in dyskinetic segments of the ventricle or thinning of the infarct leading to aneurysms. Either of these consequences will significantly decrease global cardiac function. Compensatory mechanisms resulting in increased mechanical stress could lead to programmed cell death of cardiocytes in the non-infarcted myocardium, resulting in cardiac remodeling. (Cheng W, et al., “Stretch-induced programmed myocyte cell death,” J. Clin. Invest. 96: 2247-2259, 1995). Cardiac remodeling of noninfarcted myocardium has been suggested to cause ventricular dilatation which further contributes to ventricular dysfunction and the propensity for malignant arrhythmias (Beltrami C, et al., “Structural basis of end-stage failure in ischemic cardiomyopathy in humans,” Circulation 89: 151-163, 1994; and Olivetti G, et al., “Side-to-side slippage of myocytes participates in ventricular wall remodeling acutely after myocardial infarction in rats.” Circ. Res. 67: 23-34, 1990.).

The therapies according to various aspects of the invention, as illustrated variously according to the embodiments described herein, prevent the negative remodeling process of infarct related wall thinning and aneurysm formation. Congestive heart failure will be treated by the prevention of LV aneurysms and improved LV function.

Furthermore, the therapies provided by such aspects of the invention are useful for increasing wall thickness in chronic ischemic cardiomyopathy or idiopathic dilated cardiomyopathy. Increased mechanical stress leads to cardiac remodeling, ventricular dilatation and ventricular dysfunction. These factors contribute to the pathogenesis of congestive heart failure. Accordingly, these certain therapeutic aspects of the invention are beneficially utilized in a manner to improve wall thickness and function, thus preventing congestive heart failure.

According to the particular embodiments described below by reference to specific experimental examples performed, fibrin glue is in particular considered beneficial agent for such use according to various embodiments of the invention. More specifically, in certain embodiments, fibrin glue material is injected into cardiac structures such as ventricles to provide wall support. In another regard, injection of fibrin glue into cardiac tissue structures provides a molecular scaffold for cell therapy or gene therapy. Still in a further embodiment, the fibrin glue is injected in a manner which induces angiogenesis. In a further highly beneficial regard, fibrin glue is injected in a manner which provides a combination of two or all three of these benefits: wall support, molecular scaffold for cell or gene therapy, and inducing angiogenesis. As will be further developed below, fibrin glue provides various bioactive factors, such as according to certain particular fragments or bioactive sites on the fibrin molecular scaffold, which contribute to one or more of these benefits. This includes for example factors adapted to recruit endogenous cells, and providing such cellular deposition recruiting factor is considered an additional independent benefit, either alone or in conjunction with other benefits or combinations as described herein.

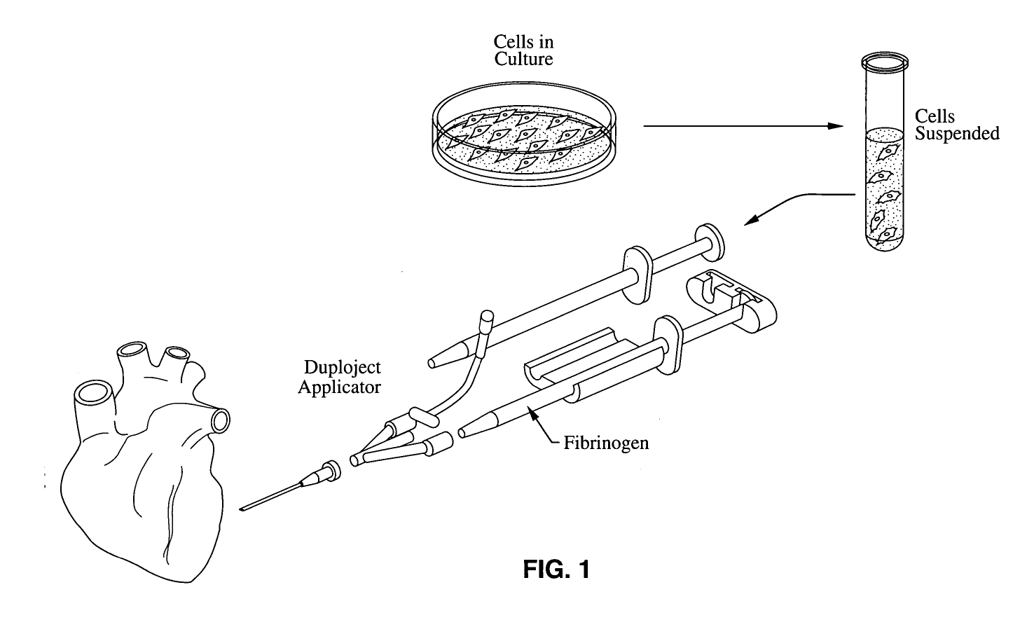

Moreover, in particular as a molecular scaffolding, the polymer or biopolymer, and in particular embodiments fibrin glue, is injected into the cardiac structure in combination with injecting cells into the structure. Such combined delivery may be in a single preparation, which may be prepared for example using a kit bedside or contemporaneous with the treatment, or in other regards may be prepared ahead of time and stored for later therapeutic use. Or, the combination may be made within a delivery catheter, such as shown in FIG. 1 and elsewhere herein described. In such combination, the cells may be combined for example with the fibrinogen or the thrombin or both components of the two-part biopolymer precursor material. Or, the injections may be done serially, either using the same delivery system and simply coupling a different fluid source, or using different delivery systems in series, each being specially adapted to deliver the cellular and polymer material, respectively.

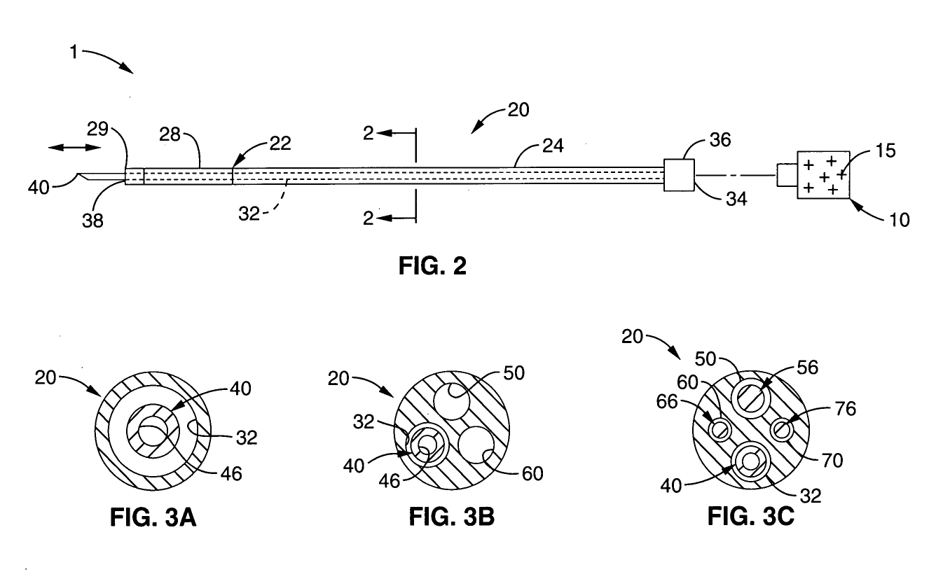

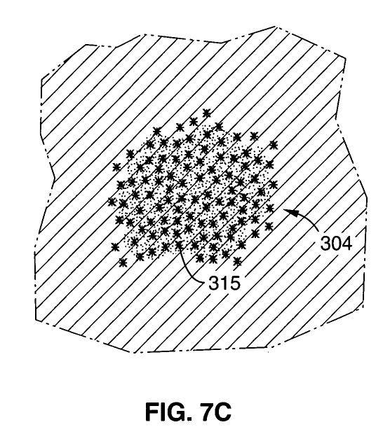

Still further, injections of the cellular and polymer material may be done simultaneously, such as through a single needle or multiple needles penetrating the structure at the same time. For example, FIG. 2 shows one embodiment of the invention that provides a cardiac treatment system 1 that includes a source of material 10 and a delivery catheter 20. Delivery catheter 20 is adapted to couple to source of material 10 and to deliver material 15 to a region of a heart in a patient, as shown for example in FIGS. 7A-C below. More specifically, according to this embodiment, delivery catheter 20 has an elongate body 22 with a proximal end portion 24, a distal end portion 28, and a lumen 32 extending therethrough between proximal and distal ports 34,38 located along proximal and distal end portions 24,26, respectively. Proximal port 34 includes a proximal coupler 36 that is adapted to couple to a coupler (not shown) on source of material 10.

Delivery catheter 20 includes a needle 40 that is adapted to extend beyond distal tip 29 of catheter 20 and into tissue and further to deliver material 15 from source 10 into such tissue. Needle 40 may be fixed relative to catheter 20, or in a beneficial variation is moveable, such as axially, as shown in FIG. 2 by axial reference arrow.

The assembly of delivery catheter 20 and needle 40, in a highly simplified form, may include simply a single lumen shaft for catheter body 20 having a single lumen 32 which slideably houses needle 40 that further includes its own delivery lumen 46 for delivering material 15 as an agent into the target tissue. This arrangement is shown for example in cross-section in FIG. 3A. Alternatively, a multi-lumen design may be incorporated, as shown in variations in FIG. 3B-C as follows.

FIG. 3B shows a cross section of a multi-lumen design with needle 40 residing within catheter lumen 32, and also further providing additional lumens 50 and 60 in catheter 20. These additional lumens may have various different functions, depending upon the particular needs.

In the particular variation shown in FIG. 3C, lumen 50 houses a pull-wire 56, whereas lumens 60 and 70 house lead wires 66 and 76. Pull-wire 56 extends between a first securement point at tip 29 and an actuator (not shown) along proximal end portion 24 that is adapted to allow for axial manipulation of pull-wire externally of the body, to thereby deflect distal end portion 28 in-vivo. For deflectable tip designs, certain other material properties are generally taken into account, such as catheter shaft design, flexibility of material chosen for shaft construction, etc., and various other substitute deflection or other manipulation designs or techniques are also contemplated. For example, rather than pull-wire, push wires may be used, or other members than wires such as polymer filaments or fibers, or torsional members. In another alternative design not shown with respect to the present embodiment, a guidewire tracking member is provided to work over a guidewire as a rail for remote positioning in-vivo.

Lead wires 66 and 76 extend between a mapping electrode, such as may be provided at tip 29 or otherwise along distal end portion 28, and a proximal electrical coupler that is adapted to couple to a mapping monitoring assembly to provide an overall mapping system with catheter 20 for determining the location for material injection to form intra-tissue scaffolding. General mapping electrode configurations, or combinations of such electrodes, may be suitable for such use according to one of ordinary skill. Moreover, the mapping electrode may be radiopaque for x-ray visualization. To this end, other radiopaque tip markers may also be deployed for such visualization, or other markers or visualization techniques may be used according to one of ordinary skill, such as ultrasound (for example either intravascular, intracardiac, or transesophageal), magnetic resonance imaging (“MRI”), or other suitable modes.

It is also contemplated that needle 40 may take many different forms, such as a relatively straight sharp-tip needle, or may be a hollow screw-shaped needle or other mechanism, such as to aid in anchoring at the desired location.

Moreover, catheter 20 may be adapted to provide delivery of needle 40 at other places than at tip 29, such as along the side wall of the elongate body of distal end portion 28 of catheter. In addition, multiple needles may be deployed such as along a length of catheter 20 in order to inject scaffolding along a prescribed length. To that end, the same needle may be used at different locations, such as delivery through different lumens to different ports along catheter 20, or multiple needles deployed simultaneously or sequentially.

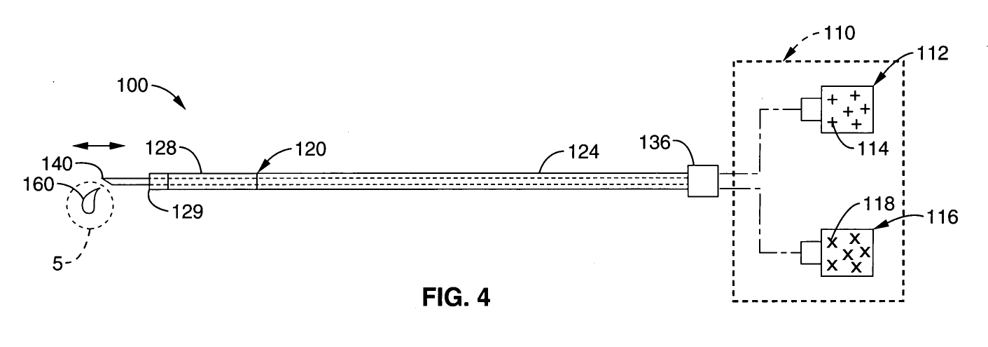

For further illustration, FIG. 4 shows a further embodiment of the invention that provides a delivery catheter 120 that is adapted to couple at proximal coupler assembly 136 along proximal end portion 124 to two sources 112,116 of two separate materials 114,118, respectively. In this regard, such combination is considered where reference to a “source of material” is elsewhere herein described, and is thus illustrated as a combination source of material 110 in FIG. 4. In this particular embodiment, the two materials 114,118 are two precursor materials to forming fibrin glue, and their combined delivery, either as the separate precursor materials that are later mixed, or in combined form mixed as fibrin glue, is hence considered a fibrin glue “agent”. Thus, “agent” in this use is intended to mean the end result, or the necessary combination of precursor material components that lead to the resultant material, though in other regards “agent” may also include the desired resulting material itself.

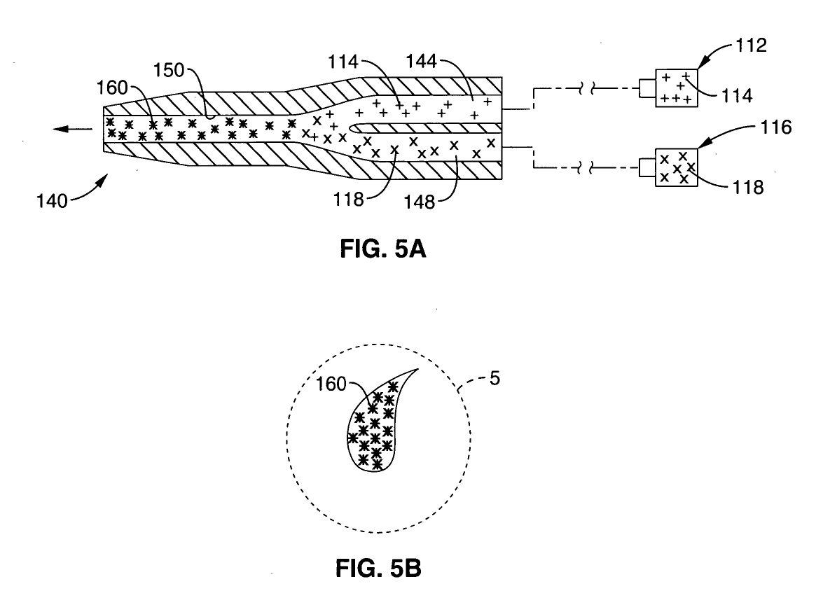

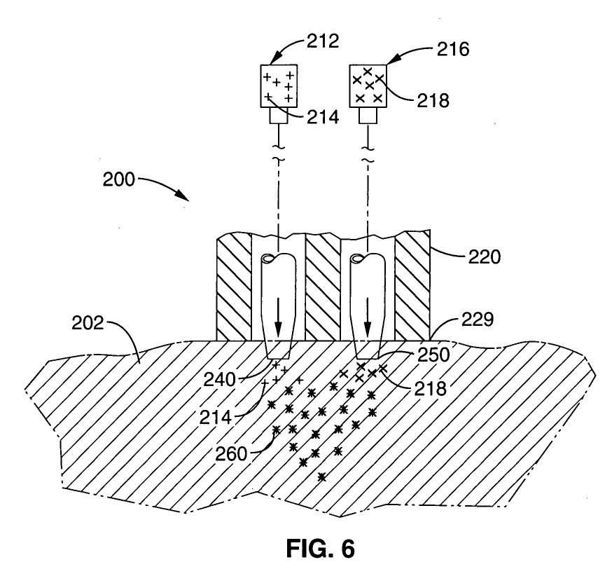

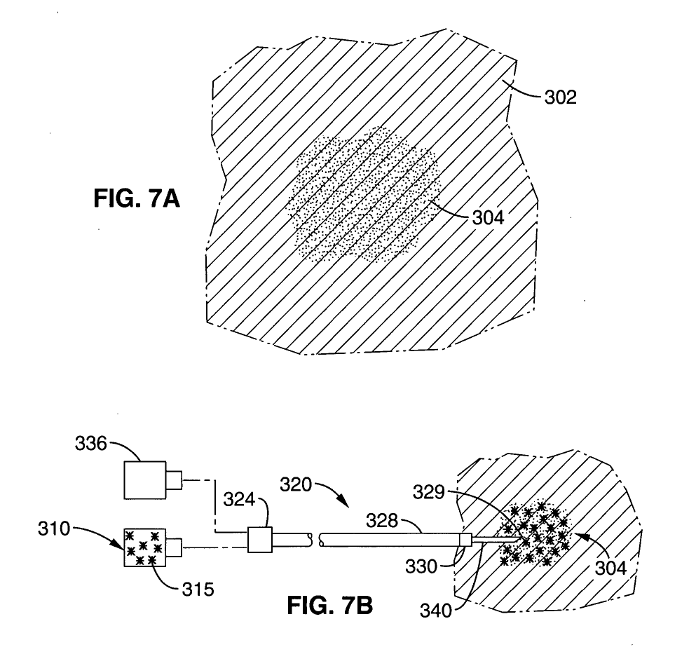

Accordingly, a system 100 as shown in FIG. 4 and by further reference to FIGS. 5A-5B, is adapted to deliver precursor materials 114,118 into the body separately, where they are therein mixed and delivered through needle 140 beyond tip 129 of distal end portion 128 into tissue as a mixed form of fibrin glue 160. An exemplary needle assembly 140 shown in FIG. 5A for accomplishing this objective delivers precursor materials 114,118 via separate lumens 144,148, respectively, that converge into mixing lumen 150 related to needle assembly 140 wherein fibrin glue 160 is formed just prior to injection via needle 140 as an injected fibrin glue, as shown in exploded view in FIG. 5B.

It is contemplated that the assembly and various components of system 100 shown by way of the embodiments in FIGS. 4-5B are illustrative, and other suitable devices may be used in order to achieve the objective of delivering two precursor materials and mixing them to form the media for injection. For example, in certain circumstances, they may be mixed prior to delivery into the distal portions of catheter 120, such as at a mixing chamber in proximal coupler 136, or prior to coupling to delivery catheter 120. To this end, one coupler may be used to couple to each of multiple sources of material for delivery, or multiple proximal couplers may be used.

Still further, more than one delivery device or injection needle may be used for each of two materials being delivered. For example, FIG. 6 shows a schematic view of a system 200 wherein a distal end 229 of catheter 220 in contact with a reference region of cardiac tissue 202. In this embodiment, two separate and distinct needles 240,250 are used to deliver each of two materials 214,218, respectively, from sources 212,216, also respectively, located outside of the patient's body. In this manner, two precursor materials are delivered separately into the tissue 202 where they mix to form fibrin glue 260 within the tissue structure. This provides the benefit of preventing unwanted clogging of the respective delivery lumen within catheter 220 during delivery to the remote in-vivo tissue location.

Further to this example, various other structures may contribute to the overall system 200, such as for catheter 220, including for example an actuator (not shown) that may be one common actuator or multiple independent actuators for advancing needles 240,250 into tissue 202, and/or otherwise injecting the materials 214,218 respectively therethrough.

In addition, the systems 100 and 200 just described are illustrated for use with fibrin glue agents that include a combination of two precursor materials. However, other materials may be substituted for use in such systems, and such systems may be appropriately modified for a particular material delivery. For example, cells may be delivered in combination with a second material according to either system 100 or 200, or as otherwise contemplated hereunder. Such second material may itself be a fibrin glue or other biopolymer agent, which may illustrate further multiples of sources and delivery lumens. For further understanding, for example the embodiment of FIG. 4-5B may be combined with that of FIG. 6 as follows. A source such as source 212 in FIG. 6 may include cells as material 214 to be delivered. However, source 216 in that embodiment may itself include two separate sources that are precursor fibrin glue agent materials, and thus needle 250 of the FIG. 6 embodiment may be of the type shown for needle 140 in FIG. 5A.

The present invention is useful for treating various cardiac tissue structures, in particular beneficial for providing injected ventricular wall scaffolds such as for example as follows by reference to FIGS. 7A-C.

More specifically, FIG. 7A schematically shows a region of cardiac tissue 302 along a ventricle that includes an infarct zone 304 or otherwise region of ischemic myocardium. As shown in FIG. 7B, the distal end portion 328 of a catheter 320 of the invention is delivered to the region at a location associated with the zone 304 such that the desired material 315 may be injected into that zone 304. This is done for example using a mapping electrode 330 provided at distal needle tip 329 and via an external mapping/monitoring system 336 coupled to proximal end portion 324 of catheter 320 outside of the body. Needle 340 is punctured into the tissue at the location, and is used to inject the desired material 315 from source 310, also coupled to proximal end portion 324 of catheter 320 outside of the body. According to this highly localized injection of the material 315 into the location of the infarct, the ventricular wall at that location is supported by the desired molecular scaffold within the tissue structure itself. According to further aspects and embodiments herein described, cellular scaffolding may also be thus provided, angiogenesis of the area may thus be created, and negative remodeling may be prevented, inhibiting progression and possible reversal of harmful cardiomyopathy. An illustrative scaffolding result according to the present embodiment is illustrated in FIG. 7C.

Each type of cardiac condition as herein contemplated is also considered to present unique circumstances, both anatomically and functionally. Each such condition thus may, in some circumstances, benefit from specially adapted delivery devices and techniques in order to provide the most appropriate respective therapy. For example, certain damaged cardiac tissue regions require precisely placed injections of the scaffolding to achieve the intended internal wall support while minimizing other possible harmful effects, such as pro-arrhythmia in surrounding non-ischemic areas. Such circumstances may benefit from specially adapted delivery devices and other considerations such as quantity of cells or other scaffolding material being delivered.

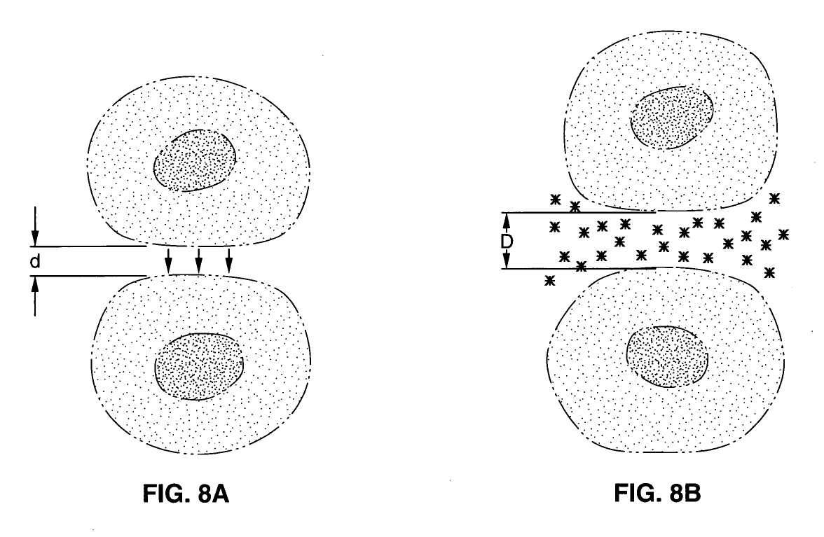

In addition to the mechanisms of action elsewhere herein described, it is further contemplated that injectable materials such as fibrin glue according to the invention may be related at least in part by its extent in the extracellular matrix and resulting physical separation of cells in the region of injection. For further illustration, FIGS. 8A-B show transition between a cellular matrix in an initial gap junction condition (FIG. 8A) having separation d, and in a post-treatment condition wherein the spacing between cells is physically separated to a larger separated distance D (FIG. 8B). These separations may be sufficient to raise the action potential to stimulate conduction between cells to such level that conduction is blocked or otherwise retarded sufficiently to potentially result in arrhythmia. Where conduction is desired along the scaffold region, further conductive additives in the artificial extracellular matrix may be added, or gap junction enhancement may be otherwise achieved such as by supporting cells modified for overexpression of Connexin 43. It is contemplated that such embodiments may incorporate, for example, cells and related gap-junction enhancing materials, and the various related methods, similar to those described in U.S. Patent Application Publication No. U.S. 2003/0104568 to Lee, or PCT Patent Application Publication No. WO 03/039344 to Lee, to the extent appropriately modified or applied in a manner consistent with this present disclosure as is apparent to one of ordinary skill. The disclosures of these references are herein incorporated in their entirety to the extent consistent with the rest of this disclosure.

It is to be appreciated that, notwithstanding various theories herein portrayed with respect to the mechanisms by which certain embodiments act, the use of certain materials and procedures to the extent they produce certain intended results are contemplated under the invention despite the actual mechanism by which the results are accomplished.

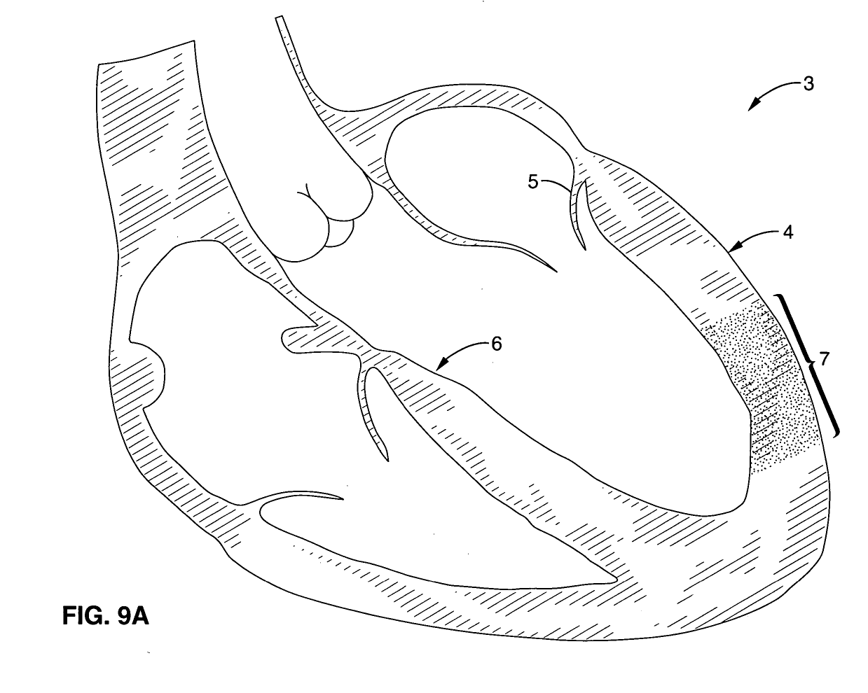

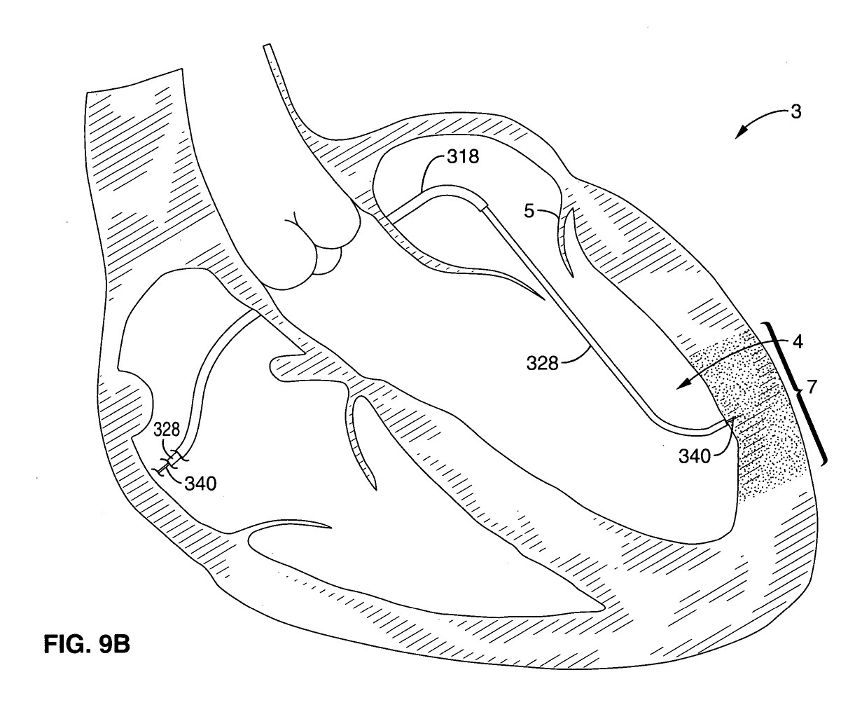

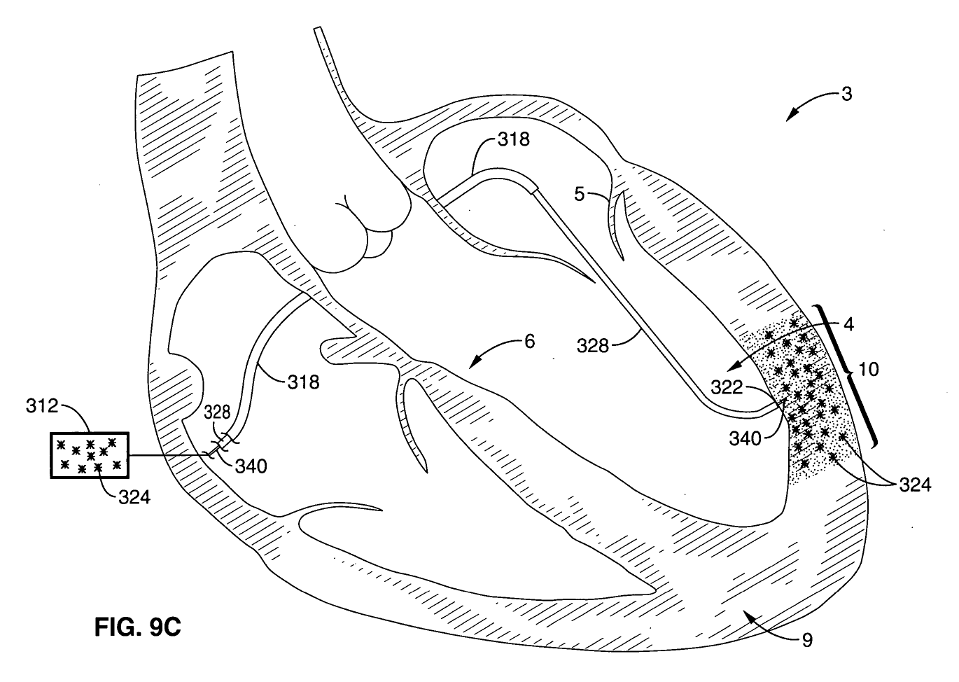

By general reference to various embodiments shown in the FIGS. 9A-C immediately following and elsewhere hereunder, certain modes of treatment are illustrated with respect to a heart 3 that is shown in various cross-sectioned views to include a left ventricle 4, mitral valve 5, inter-ventricular septum 6, and an infarct zone 7.

More specifically, FIGS. 9A-C illustrate therapeutic scaffolding treatment of an infarcted region 7 of a left ventricle 4, shown prior to treatment in FIG. 9A. Particular modes of using the present embodiment of this invention to treat such condition are illustrated in FIGS. 9B-C. As shown in FIG. 9B, an agent delivery system includes a transeptal delivery catheter 318 slideably engaged over an agent delivery catheter 328 that is further slideably engaged over a delivery needle assembly 340. Agent delivery catheter 328 is delivered into the left ventricle 4 by manipulating its proximal end portion (not shown) externally of the body via a percutaneous, translumenal approach through the venous system, and is advanced into the left ventricle 4 in a transeptal approach via transeptal delivery catheter 318 and through mitral valve 5. The distal tip 322 of the delivery catheter 328 is then positioned within the left ventricle 4 against the wall where infarct zone 7 is identified.

A source of agent 312 is coupled to a proximal end portion of the delivery catheter, as shown schematically in FIG. 9C. A volume of the scaffolding agent 324 from the source is then delivered through a delivery lumen (not shown) within the agent delivery catheter 328 and into infarct region 7, as shown in FIG. 9C. This may be accomplished using pressure alone, though in certain beneficial embodiments (e.g. shown in the present embodiment) a needle tip 340, which may in fact either integral with the delivery catheter or slideably disposed therein, is used to inject the agent 324 into the tissue. Where such a separate cooperating needle is used, the internal bore of the needle will be coupled proximally with the source of agent, as shown in FIG. 9C.

It is to be appreciated according to the embodiments herein described that one or more (e.g. an array) of electroded members may be delivered subsequent to, before, or simultaneous with delivery of agent 324 for enhancing conduction of the scaffolded region, or for mapping purposes to locate the proper injection site and pattern or area.

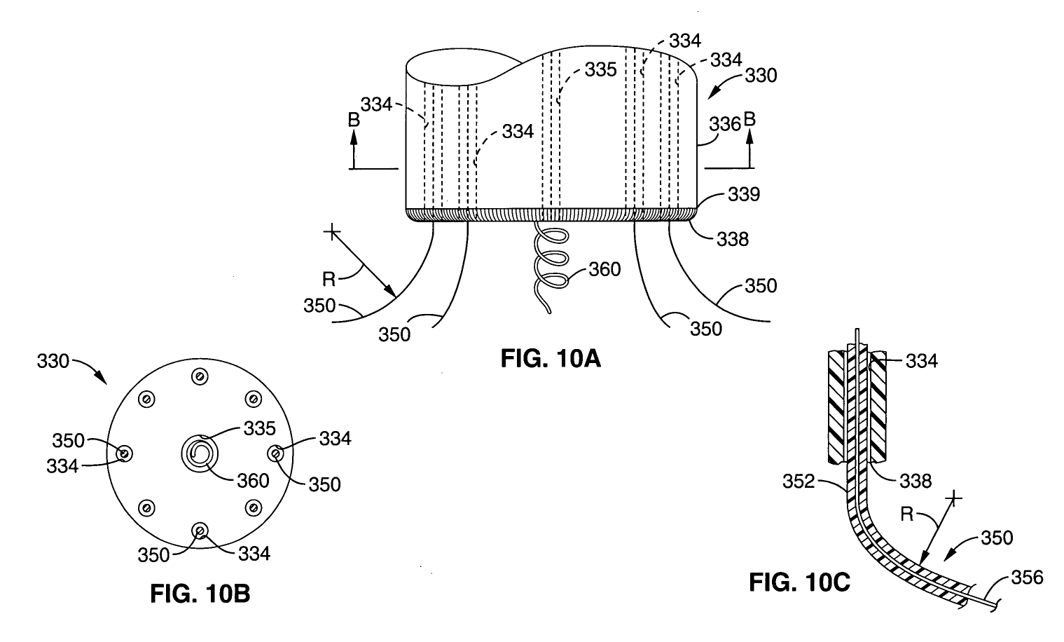

A further highly beneficial embodiment for a scaffold injection system to be used according to certain aspects of the invention, and in particular considered beneficial for endocardial delivery, is shown in FIGS. 10A-C. More specifically, delivery catheter 330 includes a body 336 with an array of lumens or passageways 334, including respective ones that are circumferentially spaced around a central lumen 335. The circumferentially spaced lumens 334 each houses a scaffolding injection needle 350, whereas the central lumen 335 houses another scaffolding injection needle 360 that forms a screw-shaped anchor adjustable in and out of that central lumen 335 for delivery to and then anchoring into the infarcted region.

Furthermore, the circumferentially spaced injection members 350 are shown according to a still more detailed embodiment in FIG. 10C to include a pre-shaped needle member 352, which may be made of nickel-titanium alloy or other superelastic, shape memory, or other suitable material, that is adapted to be housed within its respective lumen 334 during delivery of tip 338 to abut a cardiac chamber wall (e.g. ventricle), and then extendable from lumen 334 to advance into the wall for intracardiac tissue injection. Further shown is an extendable electrode member 356 that is further adjustable in and out of needle member 352. A ring electrode 339 is shown at tip 338 of scaffolding delivery catheter 330, which may be used to assist in mapping to find the optimal place for placement of the injection members 350, and/or for additional surface area for stimulation as a stimulation electrode.

In the particular embodiment shown, needle 350 has a shape-memory with a radius R that provides an angle of deflection from the long axis of the delivery assembly. It has been observed that scaffold agent injections are better performed at acute injection angles relative to the surface of the cardiac tissue structure, e.g. ventricle wall, rather than directly perpendicular injections in a normal plane to the tissue. Accordingly, in one particular variation, such angle may be for example about 30 degrees from the tissue surface—accomplished in the present illustrative example by angled deflection of the needle over its radius of memory R. Other mechanisms however may be utilized, and of course other angles of injection may be used despite the particular benefits of the embodiment just described.

Though the specific configurations shown in FIGS. 1A-C are considered beneficial, the various features such as number, placement, or specific types of elements are illustrative and other suitable substitutes may be made. For example, other numbers and corresponding placements for the circumferentially spaced injection members 350 may be used, generally desiring 2 or more injection members 350 according to the present embodiment, and generally between about 2 to about 8 injection members, or between about 2 to about 6, and in other regards between about 2 to about 4 injection members 350, in any event as considered optimal for the particular circumstances of intended use.

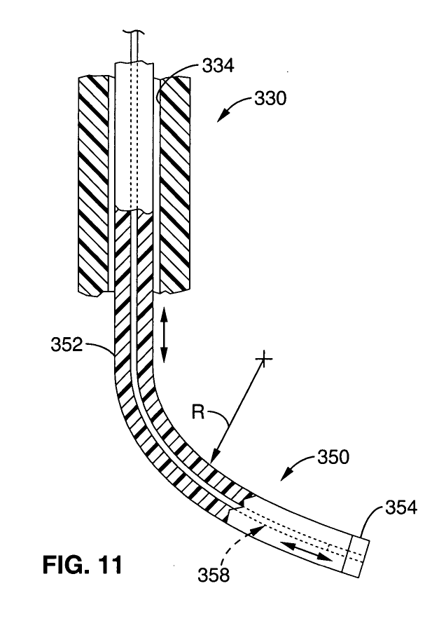

In another example shown in FIG. 11, a moveable stylet 358 is moveable within a passageway of an injection member 350 that includes a pliable shank 352 with an electrode 354 at its tip. The moveable stylet 358 is adapted to assist shank 352 during advancement through septal wall tissue to the desired location for positioning electrode at the desired region related to an infarct for scaffolding injection. Such features may be provided instead of use of the needle assembly shown and described by reference to FIG. 1C, or various modifications may be made to combine various aspects between those two approaches, including for example for a particular injection needle assembly 350, or by providing one such assembly with one design and one or more according to the other design.

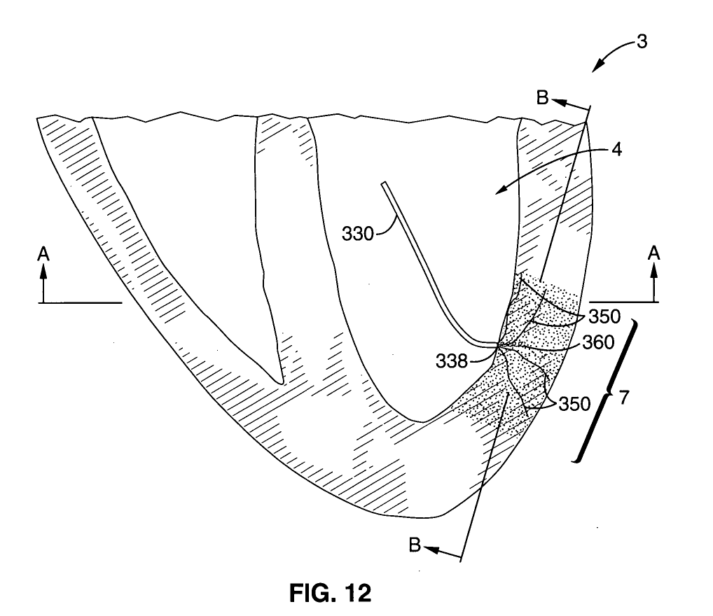

In any case, a further schematic view of the broad aspects for an arrayed scaffolding injection assembly during use is shown in FIG. 12. The array of injection members 350 is shown in angular arrangement within a transversely cross-sectioned heart for illustration, but they may share a planar orientation, such as in a plane transverse to the plane of cross-section shown for heart 3. Accordingly, anchor element 360 is located within a region of septal wall tissue that is bound by injection members 350 that have been positioned at unique respective locations around such central anchor 360 across the region. By providing scaffolding injection members 350, central injection member 360, and tip 338 as a recording electrode, the tissue bounded by injection members 350 may be substantially supported with injectate, such as for treating infarct, congestive heart failure, or cardiomyopathy.

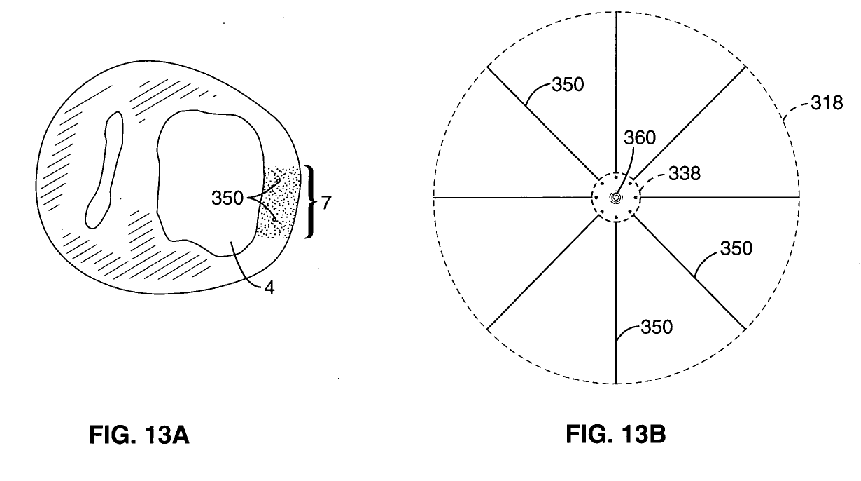

For further illustration, the orientation of such injection members 350 are shown in different planes in FIGS. 13A-B, whereas FIG. 13B is further provided with a shadowed reference to the region 7 corresponding to the tissue being stimulated. However, the circumferential arrangement shown such as in FIG. 13B corresponding to region 7 may be modified, with different shapes than circular, with different lengths of members 350, for example, or with the central area such as at anchor 360 offset within the bound region 7. In one regard, the view of FIG. 13B shows a particular view of a planar array of members 350 in two dimensions. However, they may be of modified orientation to lie in different planes such that a three dimensional volume of ventricular wall tissue is defined as the region. Still further, the array of members 350 may be further modified such that the resulting supported region 7 is instead two or more discrete regions, as further herein described.

It is to be appreciated that despite the benefits of providing intracardiac tissue support to such region 7 by elements 350, 360, and ring electrode 338 at the ventricular wall surface, it is not necessary to provide all such elements with mapping electrodes, though such arrangement may be made. Inclusion and/or removal of electrodes for any one or more of these elements, or inclusion or removal of their injection capabilities while providing for mapping, and such resulting combination arrays, are further contemplated embodiments hereof. For example, central screw injection assembly 360 may instead merely be provided as an anchor without injection and/or mapping capability. Or, it may instead be a simple needle and not necessary of the screw anchor configuration. In further examples of modifications that are contemplated, discrete injection ports may be positioned at various locations along the shanks of injection members 350 and within region 7 to ensure a thorough scaffold across the area.

It is to be appreciated therefore by one of ordinary skill that certain needle or “end-hole” injection delivery catheters (e.g. FIGS. 1-7B) may be used in certain instances to inject the scaffolding at generally a single location, such as in combination with a tip mapping electrode may be used for example. In addition, it is clear that certain more complex “needle” injection devices are herein contemplated, such as for example using screw needles with multiple ports along the screw shank, or in another example the needle devices provided herein with multiple adjacent needles intended to provide localized mixing in tissues (e.g. FIG. 6). Nevertheless, these are generally considered “point” delivery devices to the extent the intended injection is into one localized site along the plane of the cardiac tissue wall structure. In contrast, the embodiments of FIGS. 10A-13B described immediately above provide general illustration according to one of ordinary skill that such delivery may be beneficially provided along a larger region of tissue generally achievable by traditional “end-hole” injection approaches. More specifically, in order to create the necessary scaffolding to treat many varied types and extents of wall damage, it is often desired to provide the scaffolding along a substantial portion of a ventricle wall. Moreover, it is desired to match delivery of cells and other scaffolding closely to the damaged area, and thus relying on simple diffusion and other active or passive transport mechanisms from point source delivery lacks such reliability. Accordingly, the delivery catheter desired to achieve such scaffolding would be suitably adapted to inject the scaffolding material along such expansive and frequently shaped region. Such custom delivery and resulting scaffolding generally provides for more reliable and controlled impact of the therapy.

It should also be appreciated that other modifications may be made to achieve similar objectives. For example, contact members such as cages, balloons, screw or needle anchors, may be used in order to anchor a delivery assembly in place so that needles or other injection or delivery members may be then extended from a position along the delivery catheter to another location adjacent to the contact member. In another regard, it is to be appreciated that contact members may include the needles themselves, and multiple needles may be employed in a spaced fashion over a region for delivery, allowing for the injection and subsequent diffusion or other transport mechanisms in the tissue to close the gaps between scaffolds from discrete injection sites and cover the region as one example of an equivalent approach to continuous, uninterrupted contact of a delivery member over that region. In other words, “contacting” a region of tissue is considered contextual to the particular embodiment or application, and may be substantially continuous and uninterrupted contact in certain circumstances, or in others may have interruptions that are considered insignificant in the context of the anatomy or more general use.

For the purpose of further illustration, other more specific examples of delivery devices and methods that may be modified according to this disclosure to achieve certain of the various objectives of the present invention are variously disclosed in one or more of the following issued U.S. patent references: U.S. Pat. No. 5,722,403 to McGee et al.; U.S. Pat. No. 5,797,903 to Swanson et al.; U.S. Pat. No. 5,885,278 to Fleishman; U.S. Pat. No. 5,938,660 to Swartz et al.; U.S. Pat. No. 5,971,983 to Lesh; U.S. Pat. No. 6,012,457 to Lesh; U.S. Pat. No. 6,024,740 to Lesh et al.; U.S. Pat. No. 6,071,279 to Whayne et al.; U.S. Pat. No. 6,117,101 to Diederich et al.; U.S. Pat. No. 6,164,283 to Lesh; U.S. Pat. No. 6,214,002 to Fleischman et al.; U.S. Pat. No. 6,241,754 to Swanson et al.; U.S. Pat. No. 6,245,064 to Lesh et al.; U.S. Pat. No. 6,254,599 to Lesh et al.; U.S. Pat. No. 6,305,378 to Lesh; U.S. Pat. No. 6,371,955 to Fuimaono et al.; U.S. Pat. No. 6,383,151 to Diederich et al.; U.S. Pat. No. 6,416,511 to Lesh et al.; U.S. Pat. No. 6,471,697 to Lesh; U.S. Pat. No. 6,500,174 to Maguire et al.; U.S. Pat. No. 6,502,576 to Lesh; U.S. Pat. No. 6,514,249 to Maguire et al.; U.S. Pat. No. 6,522,930 to Schaer et al.; U.S. Pat. No. 6,527,769 to Langberg et al.; U.S. Pat. No. 6,547,788 to Maguire et al. The disclosures of these references are herein incorporated in their entirety by reference thereto.

To the extent these references variously relate to ablating tissue or other therapeutic uses than cell or polymer scaffolding delivery or treating the conditions contemplated hereunder, certain aspects of the respective catheter systems and therapy may be modified or otherwise per the intent and objects of this disclosure as appropriate to one of ordinary skill. For example, where ablation devices are disclosed, various related elements such as ablation electrodes, leads, transducers, optical assemblies, or the like, would be replaced with suitable elements for injecting the scaffolding materials of the type described herein. Other related elements such as ablation actuators, e.g. power sources, would be replaced with suitable sources of injectable material, and luminal structures of the delivery assemblies may be also suitably modified to provide for such injection to replace the prior modes of coupling such as electrical leads, etc. Moreover, certain aspects such as mapping and monitoring arrays and assemblies and methods maybe combined with the various features of the current embodiments according to still further modes of the present invention.

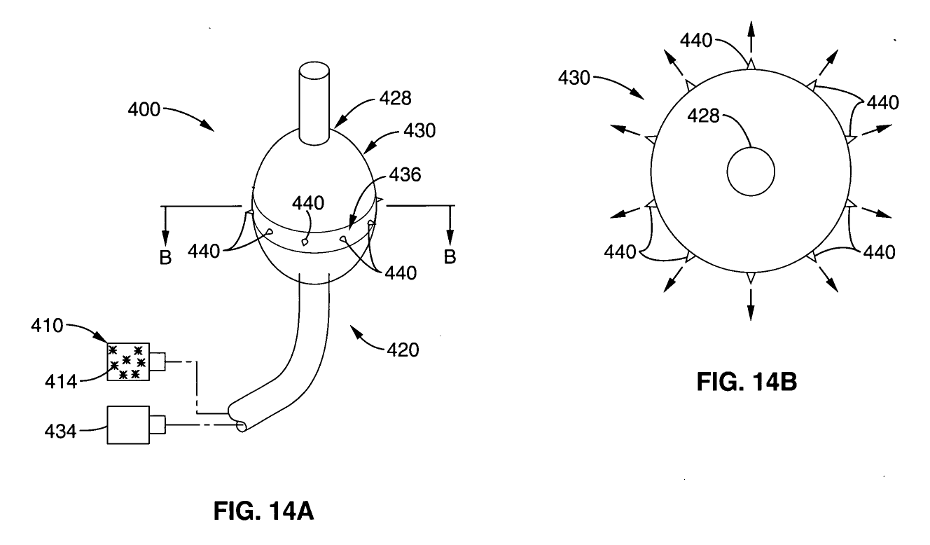

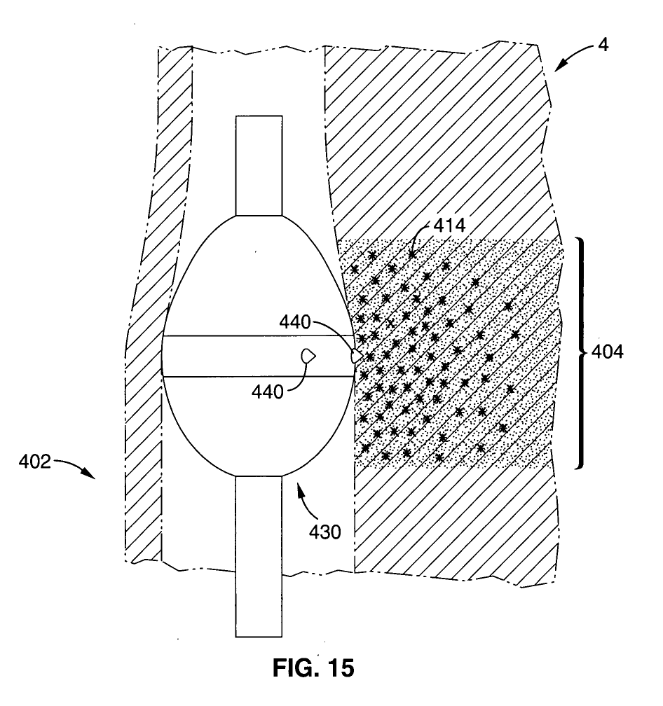

One mode of delivering injectable scaffolding material to particular regions in the heart is variously described by reference to the embodiments shown in FIGS. 14A-17B as follows.

More specifically, system 400 shown in FIG. 14A includes a delivery catheter 420 with an expandable member 430 on its distal end portion 428 and coupled to a proximal actuator 434 externally of the body. In the embodiment shown, expandable member 430 is an inflatable balloon that is coupled via catheter 420 to actuator 434 that is a source of pressurized fluid. A plurality of needles 440 are provided along one portion of balloon 430, as shown in FIG. 14A and also FIG. 14B, and couple to source 410 for delivery of scaffolding agent 414.

In certain circumstances such as treating infarcts, such injection from a device as just described is adapted to substantially isolate delivery of the scaffolding to the infarct area, or slightly larger or smaller corresponding region, wherein the desired extent of scaffolding may be customized or designed to meet a particular need. For further illustration, in the mode shown in FIG. 15, the balloon 430 is adapted to seat at the location of infarct and engage the circumferential region of vessel wall tissue with the needles 440 penetrating the infarcted tissue adjacent the vessel. By injecting the material 414 through the needles in a sufficient volume and manner, their injectate will sufficiently inject into the wall tissue and thereby form the desired scaffolding.

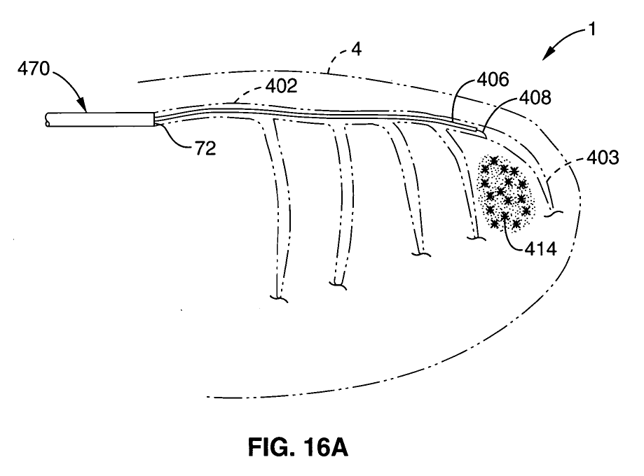

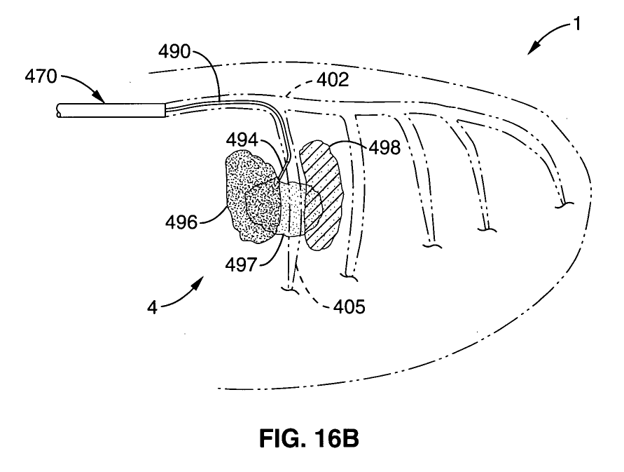

System 400 is thus particularly well adapted for forming an internal molecular scaffolding to an ischemic region of a ventricle via transvascular delivery. Other devices may also be used for such transvascular delivery of injection needles and their injectable scaffolding payload. As shown in FIG. 16, such location may be generally at a region 404 bordered by a vessel 402, such as a coronary artery or vein. For example, post re-canalization of a blocked vessel, the downstream perfusion is often directly associated with infarct. Such vessel may be used to deliver a balloon to the infarct zone, and inject through the vessel wall as shown or in other particular modes. Moreover, other routes such as coronary sinus, or again veins may be used. In addition, such balloon may be modified for use within a ventricle, using expansion to press the needled delivery portion of the balloon against the portion of wall to be injected.

It is to be appreciated that the scaffolding formed by such a devices as described by the embodiments, and in similar manner, may not be absolute or complete and still provide beneficial results. This applies in one regard to expandable member, i.e. balloon, embodiments such as just described. In one regard, transecting a portion of such a region of tissue may be sufficient to provide therapeutic scaffolding support, such as injecting “fingers” of scaffolding that function as ribs to support the region they span. In addition, such balloon designs that have insufficient needle coverage to provide for overlap between their injectates may be partially rotated one or more times for better coverage and overlap. Notwithstanding the foregoing, a complete or substantially complete injection along a damaged cardiac tissue region is a highly beneficial embodiment and believed to provide for optimal results in many cases.

For further illustration, FIG. 16A shows a schematic view of another treatment similar to that just described, wherein a delivery catheter 470 cannulates a coronary vessel 402 and delivers agent delivery device 406 to vessel 403 where needle 408 is advanced to penetrate and inject scaffolding material 414. As further illustrated by FIG. 16B, other vessels (e.g. vessel 405) may be cannulated in this manner, e.g. using guidewire tracking capabilities, and using mapping or other techniques different infarct regions may be located and treated, such as by forming sequential scaffolds 496, 497,498 with agent delivery catheter 490 and injection needle 494. By repeat injections with a repositioned needle, or multiple injections with respective needles of an array assembly, such zones overlap to treat a wider area of damage.

It is to be appreciated that the transvascular embodiments just described are illustrative and modifications may be made. For example, either balloon-assisted needles, or end-hole needle assemblies, or other equipment constructed for transvascular, extravascular scaffolding injection may be used according to the embodiments shown and discussed. Moreover, other uses of these particular devices, e.g. the balloon-based needle devices may be pursued, either according to similar designs as shown for the particular exemplary applications in the Figures, or with suitable modifications.

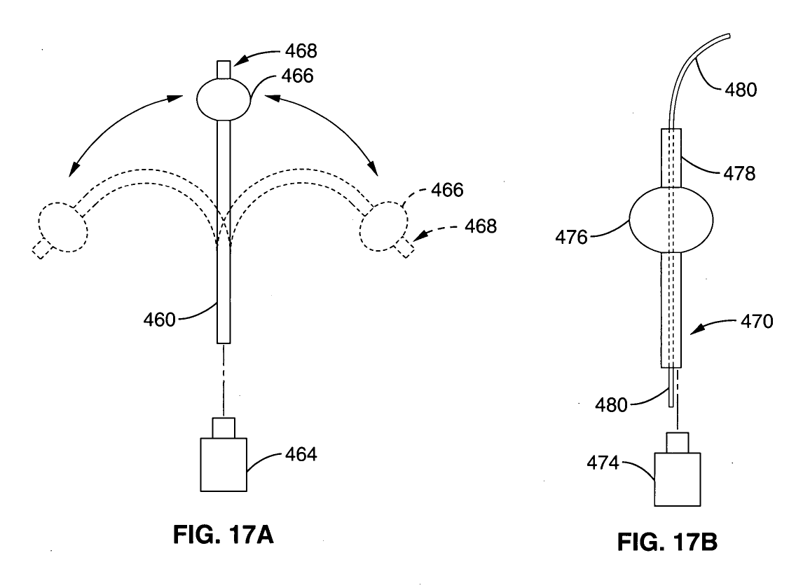

For example, various further enhancements or modifications of the device herein described by reference to FIGS. 14A-B may be made. In one particular example, a deflectable tip design shown in FIG. 17A may be used wherein catheter 460 has a distal end portion 468 with a balloon 466 that is deflectable by manipulating actuator 464. Pull wire designs for example may be employed to achieve this embodiment. In another embodiment shown in FIG. 17B, a catheter 470 has a guidewire tracking mechanism via an internal lumen that rides over a guidewire 480 so that distal end portion 478 and balloon 476 may be delivered to the pulmonary vein where the guidewire 480 is seated. Standard forms of guidewire coupling, e.g. using a hemostatic valve for example shown schematically at coupler 474 in FIG. 17B, may be used.

In further exemplary modifications, needles may be replaced by other modes for delivering the desired scaffolding agent material, such as through walls of porous membranes adapted to be engaged against tissue for delivery. Other devices than a balloon may be used as well, such as expandable members such as cages, or other devices such as loop-shaped elongate members that may be configured with appropriate dimension to form the desired area for delivery. Moreover, other regions than circular or partially circular (e.g. curvilinear) may be injected and still provide benefit without departing from the intended scope hereunder.

In still further embodiments, those particular embodiments described above for injecting scaffolding within cardiac tissue may also be combined with various pacing devices, structures, and techniques. In one regard, the needle assemblies themselves may be used for pacing the region of the heart associated with the infarct or otherwise damaged zone treated with the injected scaffold. Or, devices may be used adjunctively as different assemblies though cooperating in overall cardiac healthcare.

Further more detailed examples of devices & methods intended or otherwise adapted for pacing or other cardiac stimulation or electrical coupling are disclosed in the following issued U.S. patents: U.S. Pat. No. 4,399,818 to Money; U.S. Pat. No. 5,683,447 to Bush et al.; U.S. Pat. No. 5,728,140 to Salo et al.; U.S. Pat. No. 6,101,410 to Panescu et al.; U.S. Pat. No. 6,128,535 to Maarse. Additional examples are disclosed in the following U.S. Patent Application Publications: U.S. 2002/0035388 to Lindemans et al.; and U.S. 2002/0087089 to Ben-Haim. Still further examples are disclosed in the following published PCT International Patent Applications: WO 98/28039 to Panescu et al.; WO 01/68814 to Field; WO 02/22206 to Lee; WO 02/051495 to Ideker et al. The disclosures of all these references cited in this paragraph are herein incorporated in their entirety by reference thereto.

The present invention is described herein by reference to several highly beneficial embodiments that provide scaffolding in hearts, generally sufficient to provide therapeutic result to damaged cardiac tissue. It is to be appreciated that the terms “support”, “scaffold,” or terms of similar import, are intended to mean, in one regard, that a primary result of the intervention is providing a mechanically relevant, structural improvement, which may be with regard to one structural aspect or several. However, it is also to be considered that any material being delivered into a tissue may result in some compliance, and support and scaffold is not intended in all cases to be rigid. In another regard, it is also be appreciated that “scaffold” may be Moreover, even the therapy provided may still result in progression or maintenance of the medical conditions associated with the damage—however such may be nevertheless improved from an untreated control and still provide benefit.

In a similar regard, at some level it may be the case that most materials have some injectability and some scaffolding features to many if not most types of cells. However, a material is herein considered substantially an injectable scaffolding material with respect to cardiac cells if such material causes measurable benefit, and furthermore in most circumstances that is not outweighed by more deleterious detriment.

Moreover, it is also contemplated that while chronically improved support to damaged cardiac tissue has been observed according to certain embodiments of the invention, such chronic results may not be required to gain value and benefit from treatment in all cases.

Other specialized tools may be made for particular needs related to certain localized arrhythmias. As would be considered generally illustrated by the varied embodiments provided generally in the FIGS for example, a contact member is typically provided in the exemplary cardiac delivery system to contact the tissue at the target location and provide the required material delivery there.

Various combinations between tissue scaffolding and polymer scaffolding agent delivery are also described above by reference to the illustrative embodiments, but further combinations and sub-combinations, and modifications thereto, may be made. For example, screw needles may be adapted with a hollow lumen and used for one or the other of the cellular or polymeric agent delivery, whereas a circumferential array of needles around that central screw may be delivering the other of the two materials.

In another example, whereas FIGS. 16A-B show highly beneficial transvascular delivery of mixed scaffolding agent, respectively, into a ventricle wall, the delivery techniques may be combined for an overall result—in particular where different gauge needles or types of delivery devices are required for each component of a mixed scaffold. One precursor agent of a multiple-part scaffold may be accomplished for example transvascularly, in combination with a transcardiac approach with the other. Still further, whereas some agents may be delivered via a transcardiac delivery modality, other agents may also be delivered via the transvascular approach—each approach may provide for medical benefits at different areas of the ventricle wall, whereas their combination may provide a complete and still more beneficial medical result across the ventricle. To this end, the transcardiac approach is generally herein shown and described as the right heart system is often preferred for access. However, left ventricular transcardiac delivery of either or both of the polymer and cellular agents is also contemplated, instead of or in combination with the endo-ventricular approach (or transvascular approach). Any combination or sub-combination of these are contemplated, as should be apparent to one of ordinary skill based upon this disclosure.

Different volumes of scaffolding agent, and different numbers, sizes, patterns, and/or lengths of injection needles may be used to suit a particular need. In one regard, a prior diagnostic analysis may be used to determine the extent of the condition, location of the condition, or various anatomical considerations of the patient which parameters set forth the volume and/or pattern of scaffold agent or injection needle array to use for delivery. Or, a real time diagnostic approach may allow for stimulus or other effects to be monitored or mapped, such that the amount of agent, or distance, direction, or number of needle deployment, is modified until the correct result is achieved. Therefore, for example, the needles of such embodiments may be retractable and advanceable through tissue so that different arrangements may be tried until the damaged region is mapped and characterized for appropriate scaffolding injection.

It is further contemplated that the agent delivery and electrode embodiments, though highly beneficial in combination with each other, are independently beneficial and may be used to provide beneficial results without requiring the other.

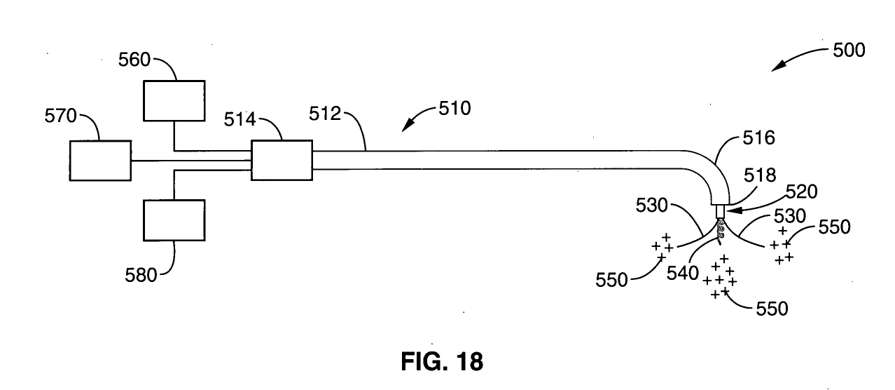

Notwithstanding the foregoing, a particular beneficial overall assembly is shown in FIG. 18. More specifically, intraventricular scaffolding system 500 is shown to include a delivery catheter 510 that cooperates to provide for both delivery of scaffolding materials 550 as well as electroded needles 530 and an anchor 540 as follows. Delivery catheter 510 has a proximal end portion 512 with a proximal coupler 514, distal end portion 516, and distal tip 518, and is an intracardiac delivery catheter adapted to deliver its contents toward the left ventricle wall from within the left ventricle chamber. Extendable from delivery catheter 510 is an inner catheter 520 with an extendable screw needle 540, and multiple spaced extendable electroded needles 530 spaced about screw needle 540. All or only some of central anchor 540, extendable electroded needles 530, and the tip of member 520 may be provided as stimulation electrodes to be coupled to energy source 560, such as via shaft 520. Moreover, all or only some of central screw 540, extendable electroded members 530, or tip of member 520, may be further adapted to deliver a volume of scaffolding agent into the region also coupled by the electroded sections, as shown at regions 550, such as via ports coupled to passageways (not shown) that are further coupled to a source of such scaffolding agent 570 (shown schematically).

This combination device is considered highly beneficial for stimulating substantial portions of the ventricle, such as for pacing and in particular treating LV wall dysfunction. As further shown in FIG. 18 and illustrative of other embodiments providing extendable elements to be driven into tissue such as in the ventricle wall, a further device 580 may be coupled to such assembly that is an actuator that either allows for automated or manual extension of the respective extendable elements. Further elements that may be provided in an overall system such as that shown in FIG. 18 at 500, or other embodiments herein, include monitoring sensors and related hardware and/or software, such as incorporated into or otherwise cooperating with an energy source such as a pacemaker/defibrillator, including for example: to map electrical heart signals for diagnostic use in determining the desired scaffolding result; and/or feedback control related to the effects of injecting the scaffolding itself, such as set points, etc.

Among the various embodiments an injectable material is described that is adapted to form a therapeutic scaffolding in cardiac tissue structures. Examples of highly beneficial materials for use according to the invention include: cells, polymers, or other fluids or preparations that provide interstitial or other forms of internal wall support, such as stiffening inter-cellular junction areas. Fibrin glue agent has been identified as a highly beneficial biopolymer for such use. Another example includes an injectable material containing collagen, or a precursor or analog or derivative thereof.

More specific modes of the invention using cells include myoblasts, fibroblasts, stem cells, or other suitable cells that provide sufficient gap junction conduction with cardiac cells to form the desired conductive coupling to the surrounding cardiac structure to provide for improved chamber conduction and contraction. In other modes, where such coupling is not achieved sufficient to provide for proper sinus rhythm through the injected region, the opposite may be desired. In other words, complete decoupling of the injected region may be preferred in order to reduce a potential “pro-arrhythmic” risk of existing, yet incomplete, contractile conduction through or from the injected zone. With further respect to cell delivery, they may be cultured from the patient's own cells, or may be exogenous and foreign to the body, such as from a regulated cell culture.

Tissue engineering techniques utilizing skeletal myoblast transplantation for myocardial repair has gained increased attention with the demonstration that skeletal myoblasts survive and form contractile myofibers in normal and injured myocardium. However, the emphasis of myocardial repair has focused on the preservation of myocardial contractility with little attention given to the effects of tissue engineering on cardiac conduction or arrhythmogenesis.

According to embodiments of the present invention using “myoblasts” together with polymeric scaffolding as a chosen living cell material to be delivered to effect a therapeutic medical result, such cells have in the past been observed to create arrhythmias when implanted into normal cardiac tissue structures, which observation is believed to result from blocking normal conduction pathways due to gap junction deficiencies between the transplanted cells and existing cardiac tissue. This has been viewed as a problem due to the prior attempts at increasing contractility and conduction with the cell therapy. In contrast, use of myoblast transplantation according to certain aspects and modes of the present invention adapts delivery of these cells in a highly localized manner at locations along infarct regions otherwise often uncoupled to the cardiac cycle, thus gap junction results between the injected and resident cells may not be substantially relevant to intended medical results.

Fibroblasts are another alternative cell of the type considered highly beneficial mode for injected internal cardiac scaffolds. In one particular beneficial regard, fibroblasts do not undergo a transition stage from proliferating to mature cells such as skeletal myoblasts. Fibroblasts therefore have a more homogeneous excitation pattern as compared to skeletal muscle. Fibroblasts' electrophysiological properties are fairly consistent from one fibroblast to the next, and are believed to be effective for consistent effects on conduction. Therefore, in one illustrative embodiment using fibroblasts to provide a scaffold to ventricular wall dysfunction or ischemia, very similar responses can be predicted between batches/injections.

Therefore the invention according to a further embodiment provides systems and methods to treat damaged myocardium using fibroblast cell transplantation in combination with injectable scaffold materials. According to a highly beneficial variation of such embodiment, such fibroblasts are autologous, typically taken from dermal samples, and are subsequently prepared appropriately and transplanted to a location within a cardiac tissue structure to facilitate scaffolding to treat cardiac injury, such as infarct, ischemia, and/or cardiomyopathy and CHF.

The invention therefore according to this beneficial embodiment uses fibroblasts from the patient's own body, and transplanting them to the area of the conduction abnormality of the heart. Fibroblasts are cells that can survive and multiply in the low oxygen environment of the scar (typically conduction abnormalities of the heart occur on the leading edge between the infarct scar tissue from an AMI and normal cardiac tissue), and also have the ability to either block or change/remodel the conduction pathway of the heart or where electromechanical coupling of the fibroblasts can be induced, creating new pathways to normalize the conduction of the heart from abnormal conduction pathways.

The disclosure of the following reference is herein incorporated in its entirety by reference thereto: Yair FELD, et. al., “Electrophysiological Modulation of Cardiomyocytic Tissue by Transfected Fibroblasts Expressing Potassium Channels: A Novel Strategy to Manipulate Exitability,” Circulation, Jan. 29, 2002 pgs 522-529.

In certain particular embodiments of the present invention, a patient's own fibroblasts are cultured and transplanted, together with injectable polymer scaffolding agent, into identified areas of damaged or otherwise dysfunctional myocardium to form a scaffolding that does not conduct contraction with or from surrounding tissues. Or, materials and methods may be employed to include the production of gap junction proteins in these fibroblast cells in order to normalize the conduction pathway via the fibroblasts' ability to electromechanically couple with the existing cardiac myocytes surrounding the injected scaffold zone.

Whereas certain broad aspects of the invention incorporate cell therapy in general for creating therapeutic mechanical scaffolding, certain more specific modes are considered also independently beneficial.

For example, in one particular such mode autologous fibroblasts are used for the treatment of infarct. Fibroblasts are a cell line that typically is associated with tissue damage (i.e., skin damage, AMI) and healing of tissue to produce scar. Activation of fibroblasts occurs in response to injury. These events cause a transition of cell types to activated phenotypes having fundamentally different biologic function from corresponding quiescent cells in normal tissue. These cellular phenotypes (arising from coordinated gene expression) are regulated by cytokines, growth factors, and down stream nuclear targets. As in the example of wound healing, fibroblasts are directed to the repair and rebuilding of tissue. Quiescent fibroblasts in normal tissue primarily are responsible for steady-state turnover of extracellular matrix, as disclosed for example in the following references: EGHBALI M, CZAJA M J, ZEYDEL M, et al., “Collagen chain mRNAs in isolated heart cells from young adult rats,” J Mol Cell Biol 1988; 20: 267-276; and POSTLETHWAITE A, KANG A., “Fibroblasts and matrix proteins; and Gallin J, Snyderman R (eds), “Inflammation. Basic Principles and Clinical Correlates,” 1999, Philadelphia: Lippincott Williams & Wilkins. The disclosures of these references are herein incorporated in their entirety by reference thereto.

Skin fibroblasts potentiate the migration to PDGF and increase collagen accumulation and MMP synthesis, and net collagen accumulation, as disclosed for example in the following reference which is also herein incorporated in its entirety by reference thereto: KAWAGUCHIY, HARA M, WRIGHT T M., “Endogenous 1 alpha from systemic sclerosis fibroblasts induces IL-6 and PDGF-A,” J Clin Invest, 1999, 103:1253-1260. This formation of collagen matrix coupled with the lack of gap junction proteins in fibroblasts creates the electromechanical isolation from cardiomyocytes. In one more particular example, a lack of electrical conduction has been observed in regions of fibroblast migration in the myocardium of patients having a previous MI.

Therefore, in certain applications where polymeric scaffolding is beneficially combined with cell therapy, fibroblasts are cells that can be utilized (and proliferated) to create electrical insulation and/or reduction of electrical conduction in regions in the myocardium that present as the arrhythmogenic foci of abnormal conduction pathways.

Fibroblasts can be biopsied from many tissues in the body (lungs, heart, skin) isolated, amplified in culture, and introduced (via injection, graft delivery, grafting, with a polymetric carrier or backbone) into a region of the heart where there is a need to reduce the conduction, isolate an arrhythmic pathway, or isolate an arrhythmogenic focus in the cardiovascular system including pulmonary veins, atria and ventricles, and atrial appendage.

Further more detailed examples of certain aspects related to cell therapy with myoblasts and/or fibroblasts as related to medical treatments are variously disclosed in the following publication references: SUZUKI, Ken et al., “Overexpression of connexin 43 in skeletal myoblasts: Relevance to cell transplantation to the heart,” J. Thorac Cardiovasc Surg 2001; 122:759-66, MURRY, Charles E. et al., “Muscle Cell Grafting for the Treatment and Prevention of Heart Failure,” J Cardiac Failure 2002; 8:6 S532-541; LONG, Carlin S. et al., “The Cardiac Fibroblast, Another Therapeutic Target for Mending the Broken Heart?” J Mol Cell Cardiol 34, 1273-1278 (2002). The disclosures of these reference are herein incorporated in their entirety by reference thereto.

Cell therapy for treating damaged myocardium according to various present embodiments is considered one mode (though highly beneficial) of a still broader aspect of the invention which provides a means for enhancing cardiac wall support by modifying the underlying cardiac tissue structure itself, more specifically associated with the cardiac chambers. This aspect provides immense benefit in providing the intended therapy without many of the other side effects and shortcomings of other conventional techniques for forming scaffolding implants, such as in particular using external “sock” or other constraint implants.

For example, tissue erosion and other substantial scarring responses that may be predicted form some other conventional constraint modalities is substantially avoided. This has particular benefit for example in preventing occlusion of externally located coronary blood vessels.

In addition, cell therapy is generally accomplished in a highly localized manner, whereas many scaffolding techniques suffer from requirements to support an entire portion of the heart well beyond the damage.

Accordingly, the present invention contemplates a broad scope with respect to providing therapeutic mechanical scaffolding directly affect the LV wall's own expansion characteristics, treating LV wall dysfunction without externally constraining the wall from expansion. As such, other suitable modes than cellular or polymeric agent therapy are contemplated according to this aspect of the invention.

In general, a “polymer” is herein defined as a chain of multiple units or “mers”. Fibrin glue for example contains polymerized fibrin monomers, and is further herein considered an illustrative example of a biopolymer since its components are biological.

Fibrin glue is an already FDA approved biomaterial that is routinely used as a surgical adhesive and sealant. This biopolymer is formed by the addition of thrombin to fibrinogen. Thrombin in a kit is an initiator or catalyst which enzymatically cleaves fibrinogen which alters the charge and conformation of the molecule, forming a fibrin monomer. The fibrin monomers then proceed to aggregate forming the biopolymer fibrin. After combination of the two thrombin and fibrinogen components, the solution remains liquid for several seconds before polymerizing. Fibrin glue agent, either immediately following mixture of the precursor materials, or by delivering the materials separately to mix in-situ, is therefore adapted to be delivered to the myocardium via injection catheters or other injectors, thus requiring only a minimally invasive procedure. It is also biocompatible and non-toxic, without inducing inflammation, foreign body reactions, tissue necrosis or extensive fibrosis.

Native fibrin is highly involved in wound healing and acts as the body's natural matrix for angiogenesis. Endothelial cells migrate through the fibrin clot via alphaᵥbeta₃ integrin binding to RGD motifs in fibrin. Production of plasmin at the location of migrating endothelial cells degrades the fibrin matrix. This decrease in fibrin density allows for capillary tube formation. As the cells migrate through the less dense fibrin, they interact with residues on the beta-chain of fibrin via vascular endothelial cadherins and promote capillary morphogenesis. In addition to providing a matrix for endothelial cell migration and capillary tube formation, fibrin also acts as a sustained release reservoir for several growth factors and fibrinolytic enzymes. A degradation product of fibrin, fibrin fragment E, is also characterized and observed to: induce angiogenesis; stimulate proliferation, migration and differentiation of human microvascular endothelial cells; and stimulate migration and proliferation of smooth muscle cells. Fibrin glue is also believed to upregulate or release various growth factors, which may recruit other cells into the infarct or inhibit the processes of LV expansion. Fibrin glue has been observed to induce fibroblast migration and may cause recruitment and proliferation of fibroblasts in the infarct, resulting in a thicker infarct wall. It is also possible that injection of fibrin glue results in recruitment of stem cells from the bone marrow, which may aid in new vessel development.

Further more detailed examples of fibrin glues that may be useful according to various aspects of the present invention are disclosed in the following reference: Sierra, D H, “Fibrin sealant adhesive systems: a review of their chemistry, material properties and clinical applications.” J Biomater Appl. 1993; 7:309-52. The disclosure of this reference is herein incorporated in its entirety by reference thereto.

According to still a further embodiment of the invention, a preparation of living material, such as for example cells, in combination with a non-living material is delivered into cardiac tissue structures to form a scaffolding there. In one further more detailed embodiment, the polymeric material is adapted to enhance retention of the cells being delivered into the location where the scaffolding is to be formed. In another regard, the polymeric material is adapted to further contribute to forming the scaffolding, such as by providing internal wall support via the polymerized chain of material within the region.

One particular example of a material that provides significant benefit in such combination with cellular therapy is fibrin glue. More specifically, fibrin glue has been observed to provide enhanced retention of cells such as myoblasts that are injected into cardiac tissue in order to treat damaged cardiac structures, such as infarct regions of a heart, as further developed by reference to one of the Examples below.

Notwithstanding the significant benefit of using fibrin glue in combination with cell delivery for treating cardiac arrhythmias, other suitable materials having beneficial effects in such combination are also contemplated, such as other polymers or molecular scaffolds or materials that intervene sufficiently to inter-cellular gap junctions or otherwise impact the extracellular matrix in cardiac tissue structures to substantially enhance function and/or support of a damaged wall structure. Moreover, collagen or precursors or analogs or derivatives thereof are further considered useful for this purpose, either in addition or in the alternative to fibrin glue.

Embodiments of injectable scaffolding material according to the invention may include primarily or only one injectable scaffolding material, or may include combinations of materials. For example, embodiments of injectable scaffolding material that includes cells may include other materials, such as fluids or other substrates to provide the cells in an overall preparation as a cellular media that is adapted to be injected, such as in particular through a delivery lumen of a delivery catheter. In one particular example that has been observed as useful, the injectable scaffolding material may include skeletal myoblasts or other suitable substitute cells in combination with a biopolymer agent such as fibrin glue agent, which may itself be provided as two precursor materials that are mixed to form fibrin glue that assists in forming a scaffold when delivered with cells at the desired location within the heart.

Notwithstanding the substantial benefit that may be gained from such specialized tools and techniques to meet particular needs as described herein, such particular modes for forming injected intercardiac wall scaffolds, or otherwise conducting cell therapy for treating or preventing cardiacmyopathies or ischemic conditions, are not to be considered limiting to the various broad aspects of the present invention.

For example, it is to be appreciated that fibrin glue expresses several different modes of beneficial bioactivity that each provides or enhances particular therapeutic results of the fibrin as an injected wall scaffold. Accordingly, the fibrin agent itself is an illustrative mode of such bioactive features as broader aspects having independent value (despite the additional value from the various combinations of features). In one regard, fibrin includes RDG binding sites which have been observed to increase affinity of cells into the area, including cell delivered with the fibrin or recruited into the area. In addition, fibrin includes a fragment “E” which has been observed to induce angiogenesis. Each of these represents an independent benefit of fibrin glue as a scaffold for cell therapy, and their combination is in particular further beneficial. For example, the cell affinity provided by the RDG binding sites allow a cellular matrix to form within the scaffolding at an injected region, whereas the angiogenesis from the fragment E allows for longevity and viability of the cellular matrix via induced blood supply. This is in particular beneficial for example in applications injecting the scaffolding into ischemic myocardium or to treat cardiomyopathy such as in CHF therapy, enhancing the ventricular wall while preventing negative remodeling that would otherwise progress without the long-term cell viability in the area.

Accordingly, the fibrin glue is to be considered illustrative of the features which provide these benefits, and other modifications may be made in further embodiments providing other injectable compounds for similar activities. For example, injecting a material into tissues as described and that express RDG binding sites in a resulting injected scaffold is a broad aspect of the invention illustrated but not limited to the particular beneficial embodiment of fibrin glue. In another example, injecting a polymer agent into cardiac tissue in a manner which induces angiogenesis is another broad aspect illustrated by the fibrin glue but not necessary limited to that particular beneficial embodiment in all cases. In particular, modifications of the detailed embodiments may include other molecular forms which provide fragment E than specifically via fibrin molecules. Still further, the combination of RDG binding activity (or other cellular affinity factors) and fragment E (or other angiogenic factors) may be achieved in other manners than specifically via fibrin without departing from such various broad aspects of the invention.

Notwithstanding the foregoing statements intended to remove the limitation of fibrin glue from certain broad aspects of the invention, it is nevertheless to be appreciated that fibrin glue does provide tremendous value and benefit in its own regard, such as by individually providing the combination of features and benefits just described as an injectable scaffold agent.

Other polymers or molecular scaffolds or materials, which may be injectable themselves or in the form of precursor agents, are briefly described as follows. Several synthetic polymers, such as polyethylene oxide (“PEO”), PEO-poly-l-lactic acid (“PLLA-PEO block copolymer”), poly(N-isopropylacrylamide-co-acrylic acid) (“poly(NIPAAm-co-Aac)”), pluronics, and poly-(N-vinyl-2-pyrrolidone) (“PVP”) may be adapted to provide artificial extracellular matrices for transplanted cells. Various biologic polymers such as alginate, collagen, and of course fibrin glue, may be prepared in a manner for use as injectable scaffolds in certain settings. Benefits of each of these polymers include that they may be injected into the desired location without the need for more invasive implantation.

In one more specific example, PEO is generally considered biocompatible and is known not to react with proteins and most biologic macromolecules. It is injectable, though larger needles such as 22 gauges are generally to be used for this material. According to another example, PEO-PLLA-PEO block copolymers are also generally considered biocompatible and biodegradable. However, formulations with this compound will typically undergo gel solution transitions around about 45° C., and thus are typically to be injected at temperatures above body temperature. A respective treatment system would in such circumstance generally also include a heater assembly. Poly(NIPAAm-co-AAc) gels also undergo gel solution transitions, which gels generally remain liquid at room temperature and solidify at body temperature. In order to have a mechanically stable gel, larger gauge needles may also be particularly useful. Pluronics are also known to be generally biocompatible, but are not typically considered biodegradable. They remain liquid at temperatures lower than 4° C., and thus catheter delivery may also further include active cooling and/or insulation along the catheter to provide and maintain the material at such temperatures until delivered. PVP is a material that may be injected through smaller gauge needles such as 30 gauge. It is also generally non-antigenic and non-toxic; however, it is generally not considered biodegradable. Alginate gels are typically linked together by calcium ions, which will dissociate and render the gel mechanically unstable over a period of time. They are also generally considered non-biodegradable and have been observed to be immunogenic in certain settings. Collagen gels are generally considered biocompatible and biodegradable, but are not typically mechanically stable.

Certain additional materials have been disclosed for use to form sponges as scaffolds for cell culture and transplantation. In one particular series of disclosures, polysaccharide sponges are intended to be applied in such a manner. However, these disclosures have not suggested suitable modifications of these structures to provide for an injectable scaffolding agent well suited for delivery via needle injection or transcatheter techniques. Nevertheless, where possible it is herein contemplated to make such modifications for injectable delivery as further aspects hereunder.

Further more detailed examples of various aspects of the materials described immediately above are provided in one or more of the following references: MERRILL E W. “Poly(ethylene oxide) star molecules: synthesis, characterization, and applications in medicine and biology,” J Biomater Sci Polym Ed, 1993; 5:1-11; PEPPAS N A, Langer R. “New challenges in biomaterials,” Science, 1994; 263:1715-20; SIMS C D, Butler P E, Casanova R, Lee B T, Randolph M A, Lee W P, Vacanti C A, Yaremchuk M J, “Injectable cartilage using polyethylene oxide polymer substrates,” Plast Reconstr Surg. 1996; 98:843-50; JEONG B, Bae Y H, Lee D S, Kim S W, “Biodegradable block copolymers as injectable drug-delivery systems,” Nature, 1997; 388:860-2; STILE R A, Burghardt W R, Healy K E, “Synthesis and Characterization of Injectable Poly(N-isopropylacrylamide)-Based Hydrogels That Support Tissue Formation in Vitro,” Macromolecules, 1999; 32:7370-7379; ARPEY C J, Chang L K, Whitaker D C, “Injectability and tissue compatibility of poly-(N-vinyl-2-pyrrolidone) in the skin of rats: a pilot study,” Dermatol Surg, 2000; 26:441-5; discussion 445-6; SMIDSROD O, Skjak-Braek G. “Alginate as immobilization matrix for cells,” Trends Biotechnol, 1990; 8:71-8; Paige K T, Cima L G, Yaremchuk M J, Vacanti J P, Vacanti C A. “Injectable cartilage,” Plast Reconstr Surg, 1995; 96:1390-8; discussion 1399-400. The disclosures of these references are herein incorporated in their entirety by reference thereto.

Various of the materials described herein are considered useful according to various of the present embodiments, either alone or in combination or blends with others, such as for example in addition or in the alternative to fibrin glue. These compounds also illustrate certain broader classes of compounds, which classes may contribute additional alternatives as would be apparent to one of ordinary skill. Moreover, the compounds listed may be delivered to tissue by delivering precursor materials to the tissue which form the intended compound in situ. For example, alginate is an illustrative form of polymerized polysaccharide which may be suitably prepared for injection and provide various of the benefits herein described. In one particular example, alginate as a polymer may be made injectable for example by varying the concentration of the polysaccharide and calcium. Such preparation, or other injectable preparation, may be thus injected into cardiac tissue structures according to various aspects described herein, again either instead of or in combination with fibrin glue or other compounds as would be apparent to one of ordinary skill.

Moreover, whereas polymers are in particular beneficial means to provide scaffolding to cardiac tissue structures and supporting cell therapy, other types of materials than polymers may be used according to various aspects of the invention and thus represent further contemplated embodiments. For example, integrin is an example of a protein which has been observed to enhance cellular binding and thus may be injected into cardiac tissue structures to provide substantial benefit to cellular tissue formation and/or retention there. For further illustration, further particular embodiments may also include integrin in combination with cell delivery, and/or in combination with others of the non-living compounds herein described as useful according one or more of the aspects of the invention.

In comparison with the foregoing list of exemplary polymers and other potential injectable scaffolding agents, it is nevertheless appreciated that fibrin glue provides a valuable and relatively unique combination of benefits in that it is generally considered biocompatible, non-toxic, and biodegradable; it may also be injected through 30 gauge needles at room or body temperature. Moreover, it provides the combination of bioactivities providing combined therapy as injectable scaffold which many other agents are not suited to provide.

It is still to be appreciated, however, that where fibrin glue or related agents are herein described, it is further contemplated that other materials such as collagen, or precursors or analogs or derivatives thereof, may also be used in such circumstances, in particular relation to forming injected scaffolding, either alone or in combination with cells.

Moreover, where a compound is herein identified in relation to one or more embodiments described herein, such as for example collagen or fibrin, precursors or analogs or derivatives thereof are further contemplated. For example, material structures that are metabolized or otherwise altered within the body to form such compound are contemplated. Or, combination materials that react to form such compound are also contemplated. Additional materials that are also contemplated are those which have molecular structures that vary insubstantially to that of such designated compounds, or otherwise have bioactivity substantially similar thereto with respect to the intended uses contemplated herein (e.g. removing or altering non-functional groups with respect to such bioactive function). Such group of compounds, and such precursors or analogs or derivatives thereof, is herein referred to as a “compound agent.” Similarly, reference herein to other forms of “agents”, such as for example “polymer agent” or “fibrin glue agent” may further include the actual final product, e.g. polymer or fibrin glue, respectively, or one or more respective precursor materials delivered together or in a coordinated manner to form the resulting material.

In addition to the description of embodiments provided immediately above, further aspects, modes, embodiments, and variations of the materials, systems, and methods of the invention are further provided by reference to certain illustrative scientific studies summarized immediately below, which are to be read in context of the totality of this disclosure as would be apparent to one of ordinary skill and are not intended to be limiting unless specifically so described and then limited to the particular aspect described.

### EXAMPLE 1

This example describes an exemplary study that was performed to examine the effects of fibrin glue, an injectable biopolymer, as an internal support and scaffold, and to confirm its improvement to cardiac function and effects on infarct wall thickness following myocardial infarction (“MI”).

1. Methods

a. Rat Myocardial Infarction Model

An ischemia reperfusion model was used in this study and was similar in various respects to that previously disclosed in the following publication which is herein incorporated in its entirety by reference thereto: Sievers R E, Schmiedl U, Wolfe C L, et al., “A model of acute regional myocardial ischemia and reperfusion in the rat.” Magn Reson Med. 1989; 10:172-81.

Female Sprague-Dawley Rats (225-250 g) were anesthetized with ketamine (90 mg/kg) and xylazine (10 mg/kg). Under sterile technique, the rats were placed in supine position and the chest was cleaned and shaved. The chest was opened by performing a median sternotomy. Keeping the landmarks of the base of the left atrium and the interventricular groove in view, a single stitch of 7-0 Ticron suture was placed through the myocardium at a depth slightly greater than the perceived level of the left anterior descending portion (LAD) of the left coronary artery while taking care not to enter the ventricular chamber. The suture was tightened to occlude the LAD for 17 minutes and then removed to allow for reperfusion. The chest was then closed and the animal was allowed to recover for 1 week.

b. Skeletal Myoblast Isolation and Culture

Myoblasts from the hind limb muscle of Sprague-Dawley neonatal rats (2-5 days old) were isolated and purified according to the following described procedure, and by further reference to the following background publication which is incorporated in its entirety by reference thereto: Rando T A, Blau H M. “Primary mouse myoblast purification, characterization, and transplantation for cell-mediated gene therapy.” J Cell Biol. 1994; 125:1275-87.

Briefly, the hind limb was harvested under Phosphate buffered saline (PBS)-Penicillin/Streptomycin (PCN/Strep) and mechanically minced. The tissue was enzymatically dissociated with dispase and collagenase (Worthington) in Dulbecco's PBS (Sigma) for 45 minutes at 37° C. The resulting suspension was then passed through an 80 um filter and the cells were collected by centrifugation. The cells were preplated for 10 minutes in order to isolate myoblasts from fibroblasts. The myoblast suspension was transferred to a collagen coated 100 mm polystyrene tissue culture dish (Corning Inc) and allowed to proliferate in growth media (80% Ham's F10C media, 20% fetal bovine serum, 1% PCN/Strep, 2.5 ng/ml recombinant human basic fibroblast growth factor) at 37° C. in a humidified atmosphere of 95% air plus 5% CO₂. Cultures were allowed to reach a confluency of 70-75% and passaged every 3-4 days (1:4 split). Further understanding of certain aspects of the myoblast material preparation is disclosed in Rando T A, Blau H M. “Primary mouse myoblast purification, characterization, and transplantation for cell-mediated gene therapy.” J Cell Biol. 1994; 125:1275-87.

c. Fibrin Glue

The fibrin glue used in this study was the commercially available Tisseel VH fibrin sealant (commercially available from Baxter). It is a two component system which remains liquid for several seconds before solidifying into a solid gel matrix. The first component consists of concentrated fibrinogen and aprotinin, a fibrinolysis inhibitor. The second is a mixture of Thrombin and CaCl₂. It is delivered through the supplied Duploject applicator, which holds the two components in separate syringes and provides simultaneous mixing and delivery, as illustrated by the exemplary embodiment in FIG. 1. The ratio of fibrinogen to thrombin components was 1:1 for this study.

d. Injections

Approximately 1 week after MI, either 0.5% bovine serum albumin (BSA) in 50 microliter PBS (control group), 50 microliter fibrin glue, 5×10⁶ myoblasts in 50 microliter 0.5% BSA, or 5×10⁶ myoblasts in 50 microliters fibrin glue was injected into the ischemic LV. Under sterile technique, the rats were anesthetized and the abdomen was opened from the xiphoid process to a left subaxillar level along the lower rib. The LV apex was exposed via a subdiaphragmatic incision, leaving the chest wall and sternum intact. Rats were randomized to either control or treatment groups and injections were made through a 30-guage needle into the ischemic LV. In the cells group, 5×10⁶ myoblasts were suspended in 50 microliter 0.5% BSA and injected into the myocardium. In the cells in fibrin group, 5×10⁶ myoblasts were suspended in 25 microliter of the thrombin component of the fibrin glue. The thrombin-cell mixture was simultaneously injected into the myocardium with 25 microliter of the fibrinogen component (FIG. 1). 25 microliter thrombin and 25 microliter fibrinogen was simultaneously injected into ischemic myocardium in the fibrin group. The diaphragm was sutured closed after suction of the chest cavity and the abdomen was subsequently closed

e. Echocardiography

Transthoracic echocardiography was performed on all animals in conscious state approximately one week after MI (baseline echocardiogram), followed by control or treatment injections 1-2 days later. Then a follow-up echocardiogram was performed approximately 4 weeks later.

The methodology of echocardiography used according to the study of this Example is generally similar to that disclosed in the following references: Youn H J, Rokosh G, Lester S J, et al., “Two-dimensional echocardiography with a 15-MHz transducer is a promising alternative for in vivo measurement of left ventricular mass in mice.” J Am Soc Echocardiogr. 1999; 12:70-5; and Nakamura A, Rokosh D G, Paccanaro M, et al., “LV systolic performance improves with development of hypertrophy after transverse aortic constriction in mice.” Am J Physiol Heart Circ Physiol. 2001; 281:H1104-12. Other reports have demonstrated the accuracy and reproducibility of transthoracic echocardiography in rats with myocardial infarcts. Further examples of transthoracic echocardiography in rats with myocardial infarcts are provided for purpose of further understanding in the following references: Scorsin M, Hagege A A, Marotte F, et al. Does transplantation of cardiomyocytes improve function of infarcted myocardium? Circulation. 1997; 96:II-188-93; and Litwin S E, Katz S E, Morgan J P, et al., “Serial echocardiographic assessment of left ventricular geometry and function after large myocardial infarction in the rat.” Circulation. 1994; 89:345-54. The disclosures of the references cited in this paragraph are herein incorporated in their entirety by reference thereto.

Briefly, the animals were shaved and placed in plastic DecapiCone restrainers (Braintree Scientific Inc.) in conscious state. A layer of acoustic coupling gel was applied to the thorax. Then the animal was placed in a prone or slightly lateral decubitus position. Echocardiography was performed using a 15-MHz linear array transducer system (Acuson Sequoia c256, Mountain View, Calif.). Care was taken to avoid excessive pressure on thorax, which could induce bradycardia. Two-dimensional images were obtained in both parasternal long and short axis views (at the papillary muscle level). Enhanced resolution imaging function (RES) was activated with a region of interest adjusted to heart size whenever possible. The gain was set for best imaging, and the compression was set at 70 dB. The images were acquired digitally and stored on magneto-optical disk (SONY EDM-230C).

Two criteria were used for imaging according to this particular experiment model. First, the short-axis view was given the criteria to demonstrate at least 80% of the endocardial and epicardial border. Second, the long-axis view was given the criteria to demonstrate the plane of mitral valve, where the annulus and the apex could be visualized. After adequate two-dimensional images were obtained, the M-mode cursor was positioned perpendicular to the ventricular anteroseptal wall (at the site of infarct) and the posterior wall, at the level of the papillary muscles. Wall thickness and left ventricular internal dimensions were measured according to the leading edge method of the American Society of Echocardiography. Fractional shortening (FS) as a measure of systolic function was calculated as FS (%)=[(LVIDd−LVIDs)/LVIDd]×100%, where LVID was the left ventricular internal dimension, d was diastole and s was systole. An echocardiographer blinded to the treatment group acquired the images and performed the data analysis. The accuracy and reproducibility of the technique have been reported in the following references previously incorporated herein by reference above: Youn H J, Rokosh G, Lester S J, et al., “Two-dimensional echocardiography with a 15-MHz transducer is a promising alternative for in vivo measurement of left ventricular mass in mice.” J Am Soc Echocardiogr. 1999; 12:70-5; and Nakamura A, Rokosh D G, Paccanaro M, et al., “LV systolic performance improves with development of hypertrophy after transverse aortic constriction in mice.” Am J Physiol Heart Circ Physiol. 2001; 281:H1104-12.

f. Histology and Immunohistochemistry

Approximately 4 weeks following the injection surgeries, the rats were euthanized with a pentobarbital overdose (200 mg/kg). The hearts were rapidly excised and fresh frozen in Tissue Tek O.C.T. freezing medium. They were then sectioned into 5 micron slices and stained with hematoxylin and eosin (H&E). A subset of hearts from the cells group and cells in fibrin glue group were stained with the MY-32 clone (Sigma), which is directed against the skeletal fast isoform of myosin heavy chain (MHC), in order to label transplanted cells. A Cy-3 conjugated anti-mouse secondary antibody (Sigma) was used to visualize labeled cells. One 250 microliter sample of fibrin glue was also fresh frozen, sectioned into 5 micron slices and stained with H&E.

g. Statistical Analysis

Data is presented as mean±standard deviation. Our lab has extensive experience with the rat myocardial infarction model and has found that infarcts have a high degree of variability, thus internal controls are implemented in order to evaluate treatment effects. Differences of fractional shortening and infarct wall thickness between measurements before and after injection were compared using a 2 tailed paired t test. Such differences were compared across treatment group using a one-way ANOVA with Bonferroni adjustment. Measurements after injection were also compared between groups using a one-way ANOVA with Bonferroni adjustment. Significance was accepted at P<0.05.

2. Results

A total of 41 rats were used in this study. Six rats died during or immediately following the infarct surgery while one rat died during the injection surgery (cells in fibrin glue group). Post-injection surgery, there was 100% survival in all groups. Final echocardiography measurements were performed on 34 rats. The control group (n=7) was injected with 0.5% BSA, the fibrin group (n=6) was injected with fibrin glue, the cells group (n=6) was injected with 5×10⁶ myoblasts, and the cells in fibrin group (n=5) was injected with 5×10⁶ myoblasts in fibrin glue.

a. Echocardiography

Echocardiography measurements were collected approximately one week post-MI (prior to injection surgery) and approximately four weeks following the injection surgery in order to determine the effects of fibrin glue, myoblasts, and a combination of the two on LV function and infarct wall thickness. As typical of post-MI progression, the control group exhibited a deterioration of LV function and thinning of the infarct wall. After four weeks there was significant deterioration in FS (P=0.0005) as well as a significant decrease in infarct wall thickness (P=0.02) (Table 1, control group). The results are generally provided in the following Table 1.

TABLE 1
Echocardiography Data

| | Before | 4 Weeks Post- | |
|---|---|---|---|
| | Injection | Injection | P |
| Fractional shortening, % | | | |
| Control group | 45 ± 8 | 22 ± 6 | 0.0005 |
| Fibrin group | 26 ± 5 | 23 ± 8 | 0.18 |
| Cells group | 29 ± 14 | 28 ± 2 | 0.89 |
| Cells in fibrin group | 42 ± 10 | 33 ± 6 | 0.19 |
| Infarct wall thickness, cm | | | |
| Control group | 0.29 ± 0.08 | 0.24 ± 0.04 | 0.02 |
| Fibrin group | 0.26 ± 0.04 | 0.23 ± 0.06 | 0.40 |
| Cells group | 0.30 ± 0.08 | 0.26 ± 0.06 | 0.44 |
| Cells in fibrin group | 0.30 ± 0.04 | 0.32 ± 0.02 | 0.43 |

In contrast, injection of fibrin glue alone, myoblasts alone, and myoblasts in fibrin glue resulted in the preservation of FS and infarct wall thickness. FS for the fibrin group, cells group, and cells in fibrin group did not significantly decrease by P-values of 0.18, 0.89, and 0.19 respectively (Table 1). In addition, there was no significant difference in infarct wall thickness for all treatment groups (P=0.40, 0.44, 0.43 respectively) (Table 1). Differences between before injection and post-injection FS and infarct wall thickness were compared among treatment groups. No significant difference was observed (P=0.52 and P=0.56 respectively), thus indicating that no single treatment was more effective than the others. A comparison of infarct wall thickness among all groups four weeks after injection demonstrates that the wall thickness of the cells in fibrin group is statistically greater than the control (P=0.009) and fibrin groups (P=0.04); however, due to the high degree of variability among infarcts as previously stated, it is more meaningful to use data comparing internal controls.

b. Histology and Immunohistochemistry

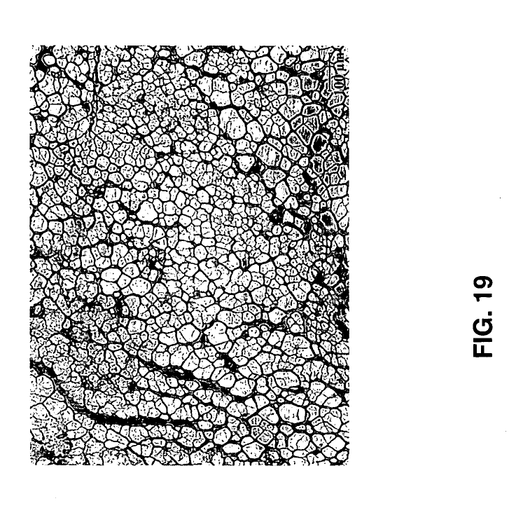

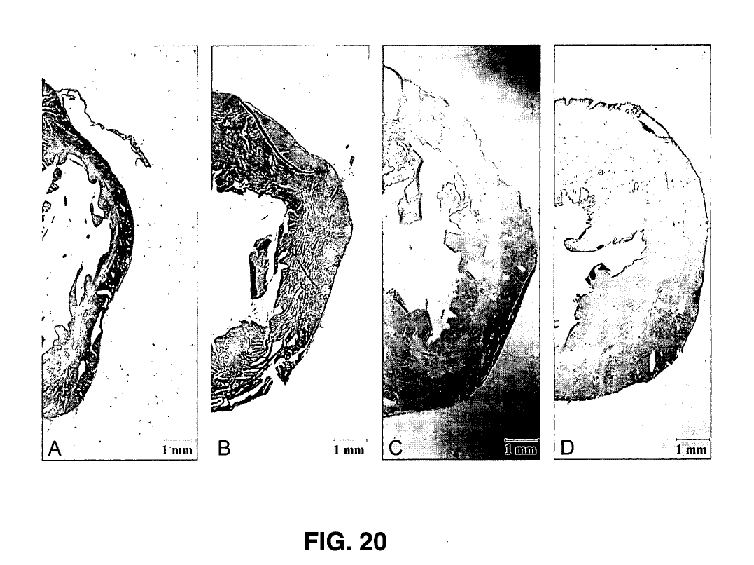

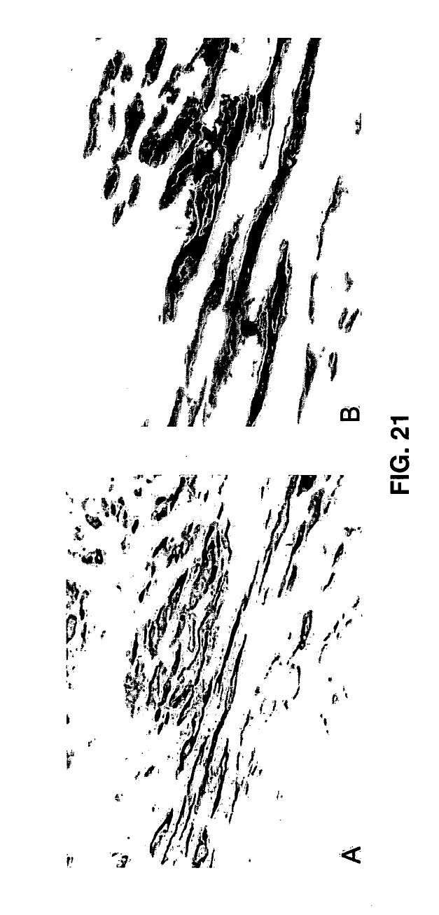

FIG. 19 illustrates both the fibril and porous nature of fibrin glue. It contains large diameter fibrils and pores (>2 micron), which is termed a coarse gel. Examination of H&E stained heart sections revealed extensive transmural MIs in all groups, as shown in FIG. 20. In the infarct region, native cardiomyocytes were replaced by fibrillar collagenous scar tissue. At four weeks after injection, the fibrin glue was completely degraded and not visible. Immunostaining for skeletal fast MHC demonstrated that transplanted cells in both the cells group and cells in fibrin group were viable four weeks post-injection and distributed throughout the infarct scar. FIG. 21 displays transplanted myoblasts in the infarct wall of a heart that was injected with myoblasts in fibrin glue. The transplanted myoblasts are aligned in a parallel orientation.

3. Discussion

Fibrin glue, though highly beneficial according to the embodiments of the study herein disclosed, is a biopolymer and thus is illustrative of other materials of similar composition or function in the environment of use that may be suitable substitutes, e.g. other biopolymers.

Fibrin is highly involved in wound healing in the body and, in conjunction with platelets, is the basis of a clot. No adverse reactions were observed upon injection into the myocardium, including no delivery of clot to or from the heart. Fibrin is resorbed by enzymatic and phagocytic pathways, thus it was expected that no traces of fibrin would remain four weeks post-injection.

The results of the present study indicate that fibrin glue is useful as a support and/or tissue engineering scaffold to prevent LV remodeling and improve cardiac function following MI. Injection of fibrin glue alone as well as injection of skeletal myoblasts in fibrin glue attenuated any decrease in infarct wall thickness and fractional shortening following MI in rats.

Injection of skeletal myoblasts alone was observed to prevent negative remodeling of the infarcted LV and deterioration of LV function. Although the exact mechanism by which myoblasts preserve LV function is unknown, it is unlikely that it is from active force generation during systole since implanted, unmodified myoblasts are not typically observed to form sufficiently conductive gap junction with surrounding cardiomyocytes. It is believed that it is the attenuation of negative left ventricular remodeling by the myoblasts that preserves cardiac function. It is believed in one regard that the myoblasts serve as a wall support by increasing stiffness, and in another regard increase wall thickness—both effects which are considered consistent with preventing negative remodeling. The data according to this study further supports this. Injection of fibrin glue alone did not produce statistically different results from the injection of skeletal myoblasts, thus suggesting that the mechanism of action of the myoblasts is by preserving wall thickness and preventing deleterious ventricular remodeling, not from active force generation.

Another previous study disclosed use of a polymer mesh for the intended purpose of acting as an external support to prevent LV dilation. Fibrin glue according to the present invention is believed to act as an internal wall support (i.e. within the wall) to preserve cardiac function. During the initial stage in MI, matrix metalloproteases are upregulated which results in degradation of the extracellular matrix (ECM). This ECM degradation leads to weakening of the infarct wall and slippage of the myocytes leading to LV aneurysm. In addition, negative ventricular remodeling has been observed to typically continue until the tensile strength of the collagen scar strengthens the infarct wall.

Fibrin glue administration during the initial stage of an infarct is observed according to this Example to prevent remodeling, and is believed to increase the mechanical strength of the infarct region before the collagen scar has had to time to fully develop. Furthermore, fibrin glue adheres to various substrates including collagen and cell surface receptors (predominately integrins) through covalent bonds, hydrogen and other electrostatic bonds, and mechanical interlocking. Therefore, it is further believed that the fibrin glue prevents myocyte slippage and subsequent aneurysm by binding to the neighboring normal myocardium. Still further, it is also believed that injection of fibrin glue results in an upregulation or release of certain growth factors such as angiogenic growth factors which are known to improve cardiac function.

In addition to providing an internal support, the data of the present Example also demonstrates that fibrin is useful as a tissue engineering scaffold in the myocardium. Injection of myoblasts in fibrin glue prevented infarct wall thinning and preserved cardiac function. The wall thickness of this group was also significantly greater than that of other groups.

The results according to the Examples presented herein indicate that fibrin glue is useful in a new and beneficial combination therapy: as a scaffold for delivering viable cells into the myocardium with substantial therapeutic results. In further embodiments therefore, cell types which produce gap junctions in recipient hearts, including fetal cardiomyocytes, adult bone marrow stem cells, or fibroblasts or myoblasts or other cell types modified to express sufficient connexins, such as Connexin-43, are thus delivered to the myocardium in fibrin glue with the aims of improving both contractility and preventing remodeling.

At least one previously disclosed reference investigated a tissue engineering approach by delivering fetal cardiomyocytes in alginate scaffolds to the surface of the myocardium and reported preservation of cardiac function. Their results are believed generally due to the transplantation of fetal cardiomyocytes and not to the external support of the scaffold due to its small size compared to the LV.

The benefits according to the various embodiments of the invention using fibrin glue as a scaffold include, in one regard, the fact that the fibrin glue is provided as an injectable agent, thus requiring only a minimally invasive procedure in humans. In addition, the cells are delivered directly into the infarcted tissue instead of simply on the epicardial surface.

The results presented according to the present Example demonstrate that preparations and use of fibrin glue according to certain aspects of the present invention provides a beneficial treatment for patients who suffer from MI. The invention thus in one aspect provides an injectable internal support and/or tissue engineering scaffold to prevent deleterious ventricular remodeling and deterioration of cardiac function. As a support, fibrin glue may be modified to tailor its mechanical properties for this particular application, which modifications are contemplated within the scope of the invention. An increase in thrombin or fibrinogen concentration results in an increase in tensile strength and Young's modulus. An increase in fibrinogen concentration will also decrease the degradation rate of the biopolymer. As a tissue engineering scaffold, fibrin glue is also capable of delivering proteins and plasmids, and further embodiments contemplated hereunder use such mechanism to deliver both growth factors, either in protein or plasmid form, and cells to the myocardium.

### EXAMPLE 2

Cellular transplantation techniques in the myocardium are limited by transplanted cell retention and survival within the ischemic or otherwise damaged tissue. This example describes an exemplary study that was performed to confirm fibrin glue's benefits as a biopolymer scaffold to improve cell transplant survival and reduce infarct size.

1. Methods

a. Rat Myocardial Infarction Model

A similar model and technique was used as described for Example 1.

b. Skeletal Myoblast Isolation and Culture

A similar method was used as described for Example 1. Cultures were routinely examined for fibroblast contamination and only populations of greater than 95% myoblasts were acceptable for injection. All injections were from the same pool of cells. Prior to injecting the rats which were sacrificed 24 hours post-injection, the myoblasts were labeled with 4′,6-diamidino-2-phenylindole (DAPI) (3 μM; Molecular Probes).

c. Fibrin Glue

The fibrin glue used in this study was similar to that described for Example 1.

d. Injection Surgeries

Similar material preparations and methods were used for this Example 2 as described for Example 1. One injection with a volume of 50 microliters was made for each animal.

e. Histology

Either 24 hours or 5 weeks following the injection surgeries, the rats were euthanized with a pentobarbital overdose (200 mg/kg). The study was concluded 6 weeks following infarction at which point the remodeling process in the rat is generally considered complete. The hearts were rapidly excised and fresh frozen in Tissue Tek O.C.T. freezing medium (Sakura). They were then sectioned into 5 micron slices and stained with hematoxylin and eosin (H&E). Five slides, equally distributed through the infarct area, were taken from each heart as a representative sample and measured for infarct size. Briefly, the infarct and LV were traced and size was determined using planimetry. Infarct size was determined as the infarct scar area divided by the total LV area as measured with SPOT 3.5.1 software (Diagnostic Instruments) and recorded as a percentage of the LV. Five additional slides from both the 24 hour cells in BSA group (n=5) and 24 hour cells in fibrin group (n=4) were examined for presence of DAPI labeled transplanted cells. The area covered by the myoblasts was traced using SPOT 3.5.1 and expressed as percentage of the infarct area. All H&E stained slides were also examined for any evidence of an immune reaction by our cardiac pathologist.

f. Immunohistochemistry

Five slides, equally distributed through the infarct area, were also taken from each heart in the 5 week BSA group (n=6), 5 week fibrin group (n=5), 5 week myoblasts in BSA group (n=5), and 5 week myoblast in fibrin group and stained with an anti-smooth muscle actin antibody (Dako; 1:75 dilution) to label arterioles. 5 slides were also taken from each heart in the 5 week myoblasts group (n=5) and 5 week myoblasts in fibrin group (n=5) and stained with the MY-32 clone (Sigma; 1:400 dilution), which is directed against the skeletal fast isoform of myosin heavy chain (MHC), in order to label transplanted cells. Sections of rat hind limb skeletal muscle were also stained with the anti-skeletal MHC antibody to serve as a positive control. Sections which were only incubated with the secondary antibody were used as negative controls. Slides were initially fixed in 1.5% formaldehyde and then blocked with staining buffer (0.3% Triton X-100 and 2% normal goat serum in PBS). Sections were incubated with the primary antibody diluted in staining buffer.

In order to visualize labeled arterioles and skeletal myoblasts, sections were incubated with a Cy-3 conjugated anti-mouse secondary antibody (Sigma; 1:100 dilution). Sections were mounted with Gel/Mount (Biomeda). Arterioles in each section were quantified using the following criteria: 1) positive for smooth muscle labeling, 2) within or bordering the infarct scar, 3) having a visible lumen and 4) a diameter ≧10 micron. The scar area was measured using SPOT 3.5.1 software and arteriole densities were calculated. Arteriole diameters were also recorded. Cell survival was determined by measuring the area covered by cells that stained positive for anti-skeletal fast MHC in each section using Scion Image (Scion) and reported as percentage of infarct area. 5 additional slides were taken from each heart in all 5 week groups. Capillaries were labeled. Slides were fixed in room temperature acetone and endogenous peroxide activity was quenched with 3% H₂O₂. Sections were incubated with biotinylated Griffonia simplicifolia lectin (GSL-1; Vector Labs). Sections were then incubated with peroxidase conjugated streptavidin (LSAB2 System, HRP, Dako), capillaries were visualized using 3,3′-diaminobenzidine chromagen solution (LSAB2 System), and sections were mounted with Gel/Mount. Five high magnification fields within the infarct of each section were chosen at random, capillaries were counted, and vessel density was calculated.

g. Statistical Analysis

Data is presented as mean±standard deviation. Cell density measurements were compared using a student's t-test. Infarct size and vessel measurements were compared using one-way ANOVA analysis with Holm's adjustment. Significance was accepted at P<0.05.

2. Results

a. Cell Retention and Survival

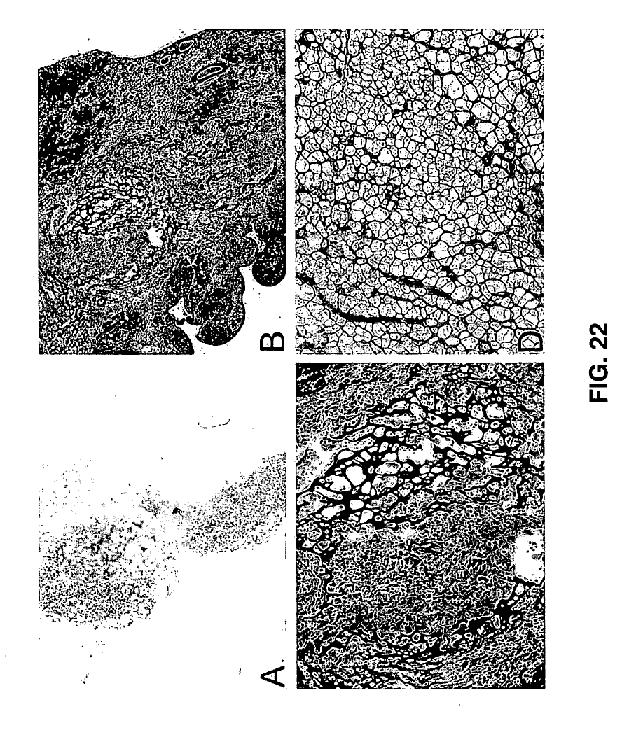

After 24 hours, the myoblast density after injection in either BSA or fibrin glue was not significantly different (P=0.85). Myoblasts injected in BSA comprised 15.8±9.2% of the infarct while myoblasts injected in fibrin glue covered 17.3±14.6%. Myoblasts transplanted in fibrin glue were found both in clumps surrounded by the fibrin matrix and dispersed within its fibrils, as shown in FIG. 22. DAPI labeled myoblasts injected in fibrin glue are found in the infarct wall, as shown in 4 times magnified view in FIG. 22A. The corresponding hematoxylin and eosin (H&E) stained section of transplanted myoblasts are surrounded by fibrin glue within the infarct scar, as shown at 4 times magnification in FIG. 22B. A higher ten times magnification H&E section displaying transplanted myoblasts in fibrin glue, as illustrated in FIG. 22C. By comparison, H&E stained section of fibrin glue ex-vivo is shown at 10 times magnification in FIG. 22D.

After 5 weeks, the myoblast density in the infarct area was significantly greater when the cells were injected in the fibrin scaffold compared to injection in BSA (P=0.03). Cells injected in fibrin glue covered 9.7±4.2% of the infarct area compared to 4.3±1.5% when injected in BSA.

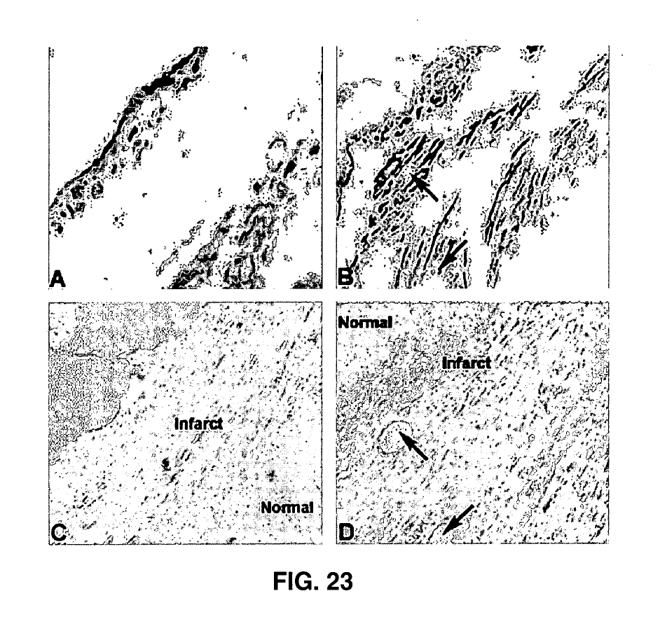

Transplanted myoblasts injected in BSA were most often found at the border of the infarct scar and not within the ischemic tissue five weeks post-injection, as shown in FIGS. 23A and 23C. In contrast, myoblasts injected in fibrin glue were found both at the border and within the infarct scar, as shown in FIGS. 23B and 23D. Cells transplanted in fibrin glue were often surrounding arterioles within the infarct scar, as shown in FIGS. 23B and 23D, see arrowheads). FIGS. 23C and 23D display the location of the normal and infarcted myocardium, thus allowing one to visualize the location of the anti-skeletal, fast MHC labeled myoblasts in FIGS. 23A and 23B respectively.

b. Histology

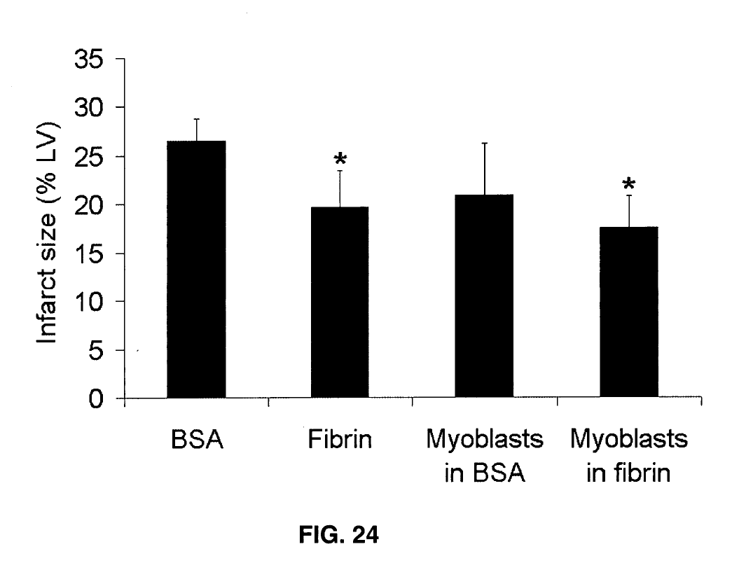

Infarct size as determined by percent of the LV was measured for each group. The infarct size in the control (BSA) group was 26.5±2.2%. There was no significant difference in infarct size between treatment groups (P=0.45); however, both injection of fibrin glue and myoblasts in fibrin glue resulted in significantly smaller infarcts (P=0.03 and P=0.003 respectively) compared to a BSA control injection. Fibrin glue reduced the infarct size to 19.7±3.8% while myoblasts in fibrin glue reduced the size to 17.5±3.4%. In contrast, myoblasts injected in BSA did not produce a statistically smaller infarct than injection of BSA (20.9±5.2%, P=0.24) (FIG. 24). Histological review of H&E stained sections from each group demonstrated that there were no significant immune reactions. The scars did contain scattered hemociderin-laden macrophages, which are evidence of prior hemorrhage, and rare mononuclear cells; however, there was virtually no active inflammation.

c. Neovasculature Formation

To assess the angiogenic potential of fibrin glue in ischemic myocardium, infarcted rat hearts injected with fibrin glue and BSA were examined for capillary density five weeks after injection. There was no significant difference between groups (P=0.64). Arterioles were labeled with anti-smooth muscle actin in both the fibrin and BSA groups to determine if fibrin glue induces arteriogenesis after 5 weeks. Even without treatment, collateral arterioles are often seen bordering the scar after MI, thus separate arteriole counts were performed on vessels within the infarct and those bordering the scar. Arteriole density for the total infarct in the fibrin group was significantly greater than that in the BSA group (P=0.004). Arterioles in the fibrin group increased to 16±1 arterioles/mm² compared to 10±2 arterioles/mm² in the BSA group. There was no difference in arteriole density bordering the infarct between the two groups (P=0.32); however there was a significant difference in the arteriole density within the scar between the fibrin and BSA groups (P=0.001). Within the infarct scar, the arteriole density following injection of fibrin glue was 13±1 arterioles/mm², compared to 10±2 arterioles/mm² for hearts injected with BSA.

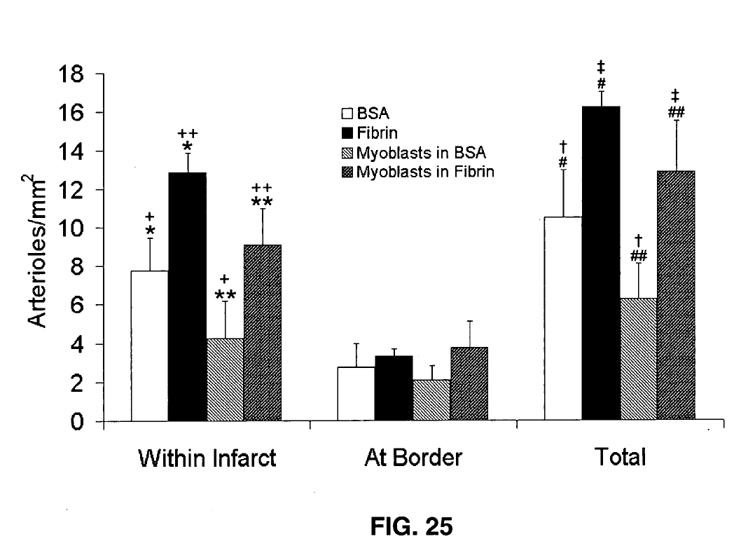

The arteriole density of the two groups including myoblasts was also calculated. Injection of myoblasts in fibrin glue significantly increased the total and within scar arteriole density compared to injection of myoblasts in BSA (P=0.007 and P=0.02 respectively). The total and within scar arteriole densities were increased to 12.9±2.6 and 9.1±1.9 arterioles per mm² compared to 6.3±1.8 and 4.2±2.0 arterioles per mm² after injection of myoblasts in BSA. There was again no difference in arterioles bordering the infarct scar (P=0.21). We also compared the BSA group to the myoblasts in BSA group and the fibrin group to the myoblast in fibrin group to determine if the addition of myoblasts affected arteriole formation. Both addition of myoblasts in BSA and fibrin resulted in a significant or near significant decrease in the total arteriole density (P=0.04 and P=0.05 respectively). Addition of myoblasts also decreased the within scar arteriole density (P=0.02 and P=0.01 respectively), as shown in FIG. 25.

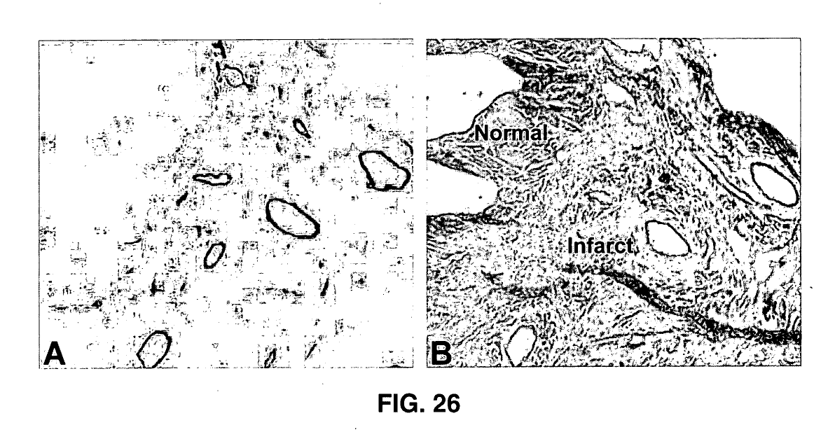

After fibrin glue injection, a large number of arterioles were found within the infarct scar, as shown in FIGS. 26A and 26B. FIG. 26A demonstrates anti-smooth muscle actin labeled arterioles visualized with a Cy3 secondary antibody. FIG. 26B has been stained with H&E and is the neighboring slide to FIG. 26A. Normal, healthy myocardium, which is denoted by its darker staining, and the infarct scar, which is denoted by lighter staining, can both be visualized in FIG. 26B. FIG. 26B demonstrates that the large number of labeled arterioles in FIG. 26A are in fact within the infarct scar.

3. Discussion

Our results indicate that cell transplant survival, but not cell retention in infarcted myocardium is enhanced by injection of cells in fibrin glue. Injection of cells in fibrin glue did not affect the amount of myoblasts in the infarct after 24 hours. These results indicate that fibrin glue does not increase cell retention. Since fibrin glue remains liquid for a few seconds, cells may continue to be squeezed out of the beating myocardium upon injection. In contrast, the area of the infarct wall covered by transplanted myoblasts after five weeks was significantly greater when the myoblasts were injected in fibrin glue, indicating that fibrin increases cell survival. Fibrin may increase cell survival by acting as a temporary extracellular matrix for the transplanted cells. Typically, cells have been injected in completely liquid formulations of saline, cell culture medium, or BSA; however, fibrin glue solidifies inside the myocardium, giving the cells a temporary semi-rigid scaffold. Fibrin glue also contains RGD motifs and binds to cell receptors (predominately integrins). Upon injection in fibrin glue, the cells are entrapped within this temporary extracellular matrix. Fibrin glue is an injectable scaffold that allows delivery of more viable cells directly into the infarct wall.

Another factor believed to contribute to the increased cell survival is that injection of fibrin glue into ischemic myocardium induced neovasculature formation. An increase in blood supply would provide a less ischemic region for the cells to thrive. Injection of cells into vascularized myocardium has been reported to increase survival compared to injection in ischemic myocardium. According to the present example, myoblasts injected in fibrin glue were often found surrounding arterioles within the infarct scar. One limitation of the animals used in this study is that they were not an inbred strain, thus graft rejection is expected to be higher. Our preliminary results with fibrin glue and myoblasts indicated that viable grafts survive in Sprague-Dawley rats. Sprague-Dawley rats represent a “worst-case” scenario for cell survival due to the increased immune reaction. If fibrin glue is capable of increasing graft size in this “worst-case”, it is to be readily appreciated that a more dramatic effect would result in an inbred strain. According to the demonstrated increase in cell transplant survival in ischemic myocardium, fibrin glue is thus confirmed as a highly beneficial modification and improvement to the standard cell transplantation procedure.

Results according to the present example further demonstrate that injection of fibrin glue alone also decreases infarct size, as was also demonstrated with myoblasts in fibrin glue. The observed increase in vasculature caused by the fibrin matrix further supports such observation. An increase in blood flow to the infarct may salvage “at risk” cardiomyocytes and produce a smaller infarct. A decrease in the area covered by the scar may also be a reduction of late infarct expansion since the infarct process is largely completed within 24 hours. As an indicator of negative LV remodeling, a decrease in late infarct expansion indicates that fibrin is capable of preventing negative left ventricular remodeling following MI in rats. Fibrin provides an internal wall support—it is considered to increase stiffness. It is also believed that fibrin affects remodeling at least in part by increasing wall thickness. Although, there was no significant difference in infarct size among treatment groups. Injection of skeletal myoblasts in BSA did not produce a statistically smaller infarct than the control, consistent with previous reports of transplantation survival problems within infarcted myocardium. This trend indicates that injected fibrin, and myoblasts in fibrin glue, is adapted to produces smaller infarcts compared to injection of myoblasts in BSA. Injection of myoblasts in BSA may not be capable of producing a large enough graft to reduce infarct size.

Fibrin glue also induced arteriole formation within the infarct scar. It is of significant benefit that fibrin glue is observed to result in arteriogenesis, since formation of solely capillaries does not necessarily indicate an increase in blood flow due to the ease of regression of vessels without smooth muscle. Fibrin was not observed in this experiment to increase capillary formation compared to injection of BSA. Injections into myocardium, in general, is believed to often induce some angiogenesis. Therefore, many different injectates may produce some non-specific angiogenic responses though generally not correlated directly with arteriogenesis. However, fibrin is beneficially confirmed according to these experiments to provide a valuable, specific arteriogenesis.

Results from this study indicate that fibrin glue may be a potential treatment for those suffering from MI. It provides, in one regard, a treatment modality that increases neovasculature formation and decreases infarct size. In another regard, it is confirmed to provide a highly beneficial method for increasing cell transplant survival in ischemic myocardium.

### EXAMPLE 3

In the study performed according to this Example 3, the use of an injectable fibrin scaffold to preserve cardiac function in a chronic MI model was demonstrated and various benefits were confirmed.

1. Methods

Methods of creation of MI, isolation and culture of skeletal cells, use of fibrin and echocardiograpy are described in Example 2.

a. Injection Surgeries

Similar injection surgery protocol over various treatment and control groups was used as described above for Example 2 and further with respect to Example 1, provided that according to this Example 3 injections were made about five weeks after myocardial infarction (MI), following completion of the remodeling process.

b. Echocardiography

Transthoracic echocardiography was performed on all animals in conscious state five weeks after MI (baseline echocardiogram), followed by control or treatment injections 1-2 days later. Then a follow-up echocardiogram was performed 5 weeks after injection (10 weeks after MI). The methodology of echocardiography used were similar to that described for Example 2.

c. Histology and Immunohistochemistry

Following the second echocardiogram (10 weeks post-MI), the rats were euthanized with a pentobarbital overdose (200 mg/kg). The hearts were rapidly excised and fresh frozen in Tissue Tek O.C.T. freezing medium (Sakura). They were then sectioned into 10 micron slices and stained with hematoxylin and eosin (H&E). All H&E stained slides were examined for any evidence of an immune reaction. Five slides, equally distributed through the infarct area, were also taken from each heart in the BSA group (n=5) and fibrin group (n=7) and stained with an anti-smooth muscle actin antibody (Dako; 1:75 dilution) to label arterioles. 5 slides were also taken from each heart in the myoblasts in BSA group (n=6) and myoblasts in fibrin group (n=5) and stained with the MY-32 clone (Sigma; 1:400 dilution), which is directed against the skeletal fast isoform of myosin heavy chain (MHC), in order to label transplanted cells. Sections of rat hind limb skeletal muscle were also stained with the anti-skeletal MHC antibody to serve as a positive control. Sections which were only incubated with the secondary antibody were used as negative controls.

Slides were initially fixed in 1.5% formaldehyde and then blocked with staining buffer (0.3% Triton X-100 and 2% normal goat serum in PBS). Sections were incubated with the primary antibody diluted in staining buffer. In order to visualize labeled arterioles and skeletal myoblasts, sections were incubated with a Cy-3 conjugated anti-mouse secondary antibody (Sigma; 1:100 dilution). Sections were mounted with Gel/Mount (Biomeda). Arterioles in each section were quantified. The scar area was measured using SPOT 3.5.1 software and arteriole densities were calculated. Cell survival was determined by measuring the area covered by cells that stained positive for anti-skeletal fast MHC in each section using Scion Image (Scion) and reported as percentage of infarct area.

d. Statistical Analysis

Data is presented as mean±standard deviation. Cell density measurements were compared using a student's t-test. 5 week and 10 week post-MI echocardiography data was compared using a paired t-test. 10 week data and arteriole density was compared using one-way ANOVA analysis with Holm's adjustment. Significance was accepted at P<0.05.

2. Results

a. Echocardiography

As typical of post-MI progression, the BSA control group exhibited a deterioration of LV function and an expansion of LV size. After ten weeks there was a significant deterioration in FS (P=0.04), a significant decrease in infarct wall thickness (P=0.01), and a significant increase in LVID during both systole (P=0.02) and diastole (P=0.01) (Table 2, control group).

Similarly, expansion of the LV was also seen in animals that were injected with myoblasts in BSA. The LVID during systole (P=0.02) and diastole (P=0.009) significantly increased five weeks after injection. The infarct wall thickness also significantly thinned (P=0.04). Injection of myoblasts in BSA was, however, capable of preserving LV function (P=0.20) (Table 2, cells in BSA group).

In contrast to the control BSA injections and injection of myoblasts in BSA, injection of fibrin glue alone preserved infarct wall thickness (P=0.86), systolic LVID (P=0.30), and LV function (P=0.68) (Table 2, fibrin group). Furthermore, injection of myoblasts in fibrin glue preserved infarct wall thickness (P=0.56), systolic LVID (P=0.31), diastolic LVID (P=0.05), and LV function (P=0.47) (Table 2, cells in fibrin group).

Although both fibrin glue alone and myoblasts injected in fibrin glue preserved LV geometry and cardiac function, at 10 weeks post-MI, animals which were injected with myoblasts in fibrin glue had significantly smaller systolic LVID (P=0.003) and significantly better fractional shortening (P=0.002) compared to injection of fibrin glue alone. At 10 weeks, animals in the cells in fibrin group also had statistically better systolic LVID (P=0.0496) and cardiac function (P=0.02) compared to animals injected with BSA. The infarct wall thickness (P=0.002), systolic LVID (P=0.01), and fractional shortening (P=0.001) of animals in the cells in BSA group were also significantly worse than those in the cells in fibrin group (Table 3).

As a control for each rat's level of excitement, the heart rate was also measured. There was no significant difference in heart rate between groups (P=0.92).

b. Histology and Immunohistochemistry

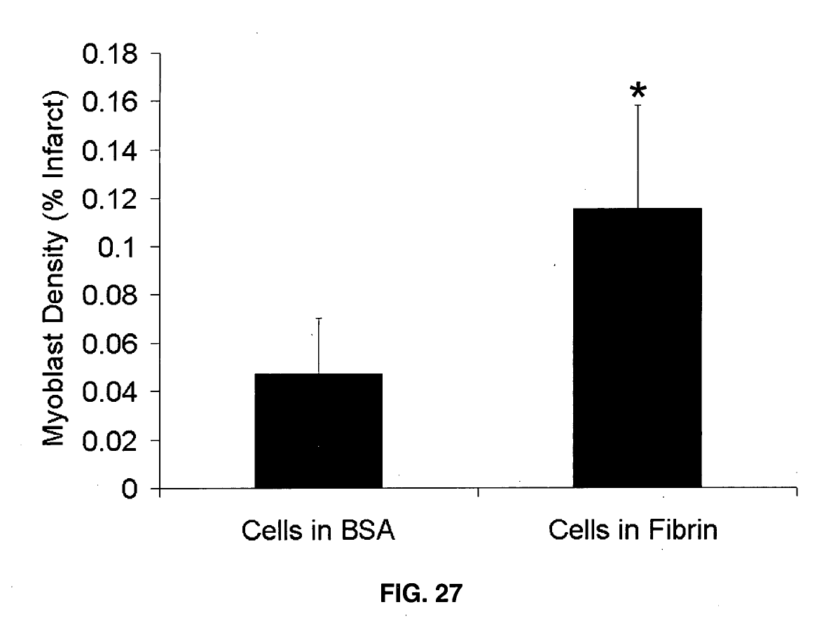

Transplanted myoblasts were labeled with anti-skeletal fast MHC to determine whether injection of cells in fibrin glue increased cell survival in the chronic MI model. After 5 weeks, the myoblast density in the infarct area was significantly greater when the cells were injected in the fibrin scaffold compared to injection in BSA (P=0.008). Cells injected in fibrin glue covered 11.5±4.3% of the infarct area compared to 4.7±2.3% when injected in BSA (FIG. 27).

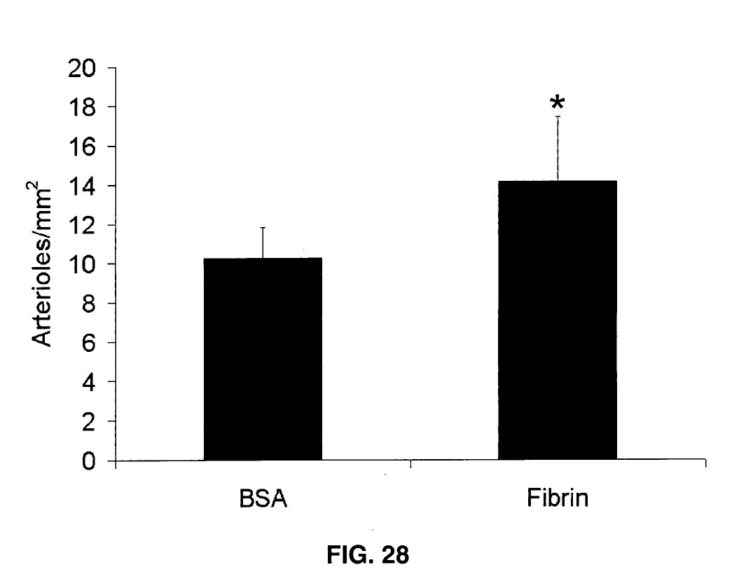

Arterioles were labeled with anti-smooth muscle actin in both the fibrin and BSA groups to determine if fibrin glue induces arteriogenesis when delivered 5 weeks after infarction. Fibrin glue significantly increased arteriole formation compared to injection of BSA (P=0.04). Arteriole density increased to 14.2±3.3 arterioles per mm² after fibrin injection compared to 10.2±1.6 per mm² after BSA injection (FIG. 28).

Histological review of H&E stained sections from each group demonstrated that there were no significant immune reactions.

Various data as results according to the experiments of the present example are provided in Tables 2 and 3 as follows:

TABLE 2
Echocardiography Data

| | Before Injection | After Injection | |
|---|---|---|---|
| | (5 Weeks Post-MI) | (10 Weeks Post-MI) | P |
| Fractional | | | |
| shortening, % | | | |
| Control Group | 52.8 ± 9.8 | 38.2 ± 13.2 | 0.04 |
| Fibrin Group | 39.9 ± 15.0 | 36.9 ± 9.7 | 0.68 |
| Cells Group | 41.2 ± 18.9 | 34.2 ± 9.2 | 0.20 |
| Cells + Fibrin Group | 67.0 ± 8.0 | 63.4 ± 6.8*† | 0.47 |
| Infarct wall thickness, | | | |
| cm | | | |
| Control Group | 0.17 ± 0.04 | 0.13 ± 0.03 | 0.01 |
| Fibrin Group | 0.12 ± 0.06 | 0.13 ± 0.05 | 0.86 |
| Cells Group | 0.14 ± 0.04 | 0.10 ± 0.03 | 0.04 |
| Cells + Fibrin Group | 0.17 ± 0.02 | 0.18 ± 0.02† | 0.56 |
| LVID systole, cm | | | |
| Control Group | 0.32 ± 0.10 | 0.48 ± 0.16 | 0.02 |
| Fibrin Group | 0.42 ± 0.14 | 0.48 ± 0.10 | 0.30 |
| Cells Group | 0.47 ± 0.20 | 0.57 ± 0.18 | 0.02 |
| Cells + Fibrin Group | 0.20 ± 0.05 | 0.24 ± 0.04*† | 0.31 |
| LVID diastole, cm | | | |
| Control group | 0.66 ± 0.11 | 0.75 ± 0.13 | 0.01 |
| Fibrin group | 0.69 ± 0.09 | 0.76 ± 0.08 | 0.04 |
| Cells group | 0.76 ± 0.13 | 0.85 ± 0.15 | 0.009 |
| Cells + Fibrin group | 0.61 ± 0.06 | 0.64 ± 0.03 | 0.05 |
| Heart Rate (beats/min) | | | |
| Control group | 480 ± 90 | 459 ± 65 | 0.29 |
| Fibrin group | 479 ± 52 | 478 ± 39 | 0.94 |
| Cells group | 490 ± 34 | 473 ± 52 | 0.27 |
| Cells in fibrin group | 499 ± 35 | 474 ± 26 | 0.13 |

*P < 0.05 vs. 10 week post-MI BSA control
†P < 0.05 vs. 10 week post-MI fibrin group and cells in BSA group

TABLE 3
10 Week Post-MI Comparisons

| Cell in Fibrin Glue | Fractional | Infarct Wall | LVID | LVID |
|---|---|---|---|---|
| vs. | Shortening | Thickness | Systole | Diastole |
| Control group | 0.02 | 0.05 | 0.0496 | 0.47 |
| Fibrin group | 0.002 | 0.24 | 0.003 | 0.08 |
| Cells group | 0.001 | 0.002 | 0.01 | 0.07 |

3. Discussion

The results of this study indicate that fibrin glue and moreover skeletal myoblasts in fibrin glue may be an alternative treatment for ischemic cardiomyopathy induced heart failure. Injection of fibrin glue alone and myoblasts in fibrin glue preserved LV geometry and cardiac function five weeks after injection, whereas myoblasts in BSA were unable to preserve infarct wall thickness and LV size. In addition, at 10 weeks post-MI, the fractional shortening and LVID during systole of the myoblasts in fibrin group was significantly better than the control, fibrin alone, and myoblasts in BSA groups. Following injection of myoblasts in fibrin, the infarct wall was also significantly thicker compared to injection of fibrin alone or myoblasts in BSA. These results confirm that both fibrin glue and a combination of myoblasts in fibrin glue are useful to prevent a deterioration of cardiac function for those suffering from a chronic MI.

The fibrin scaffold provides an internal support to prevent LV expansion and prevents a decline in cardiac function. Fibrin glue solidifies inside the myocardium and provide an internal wall support believed preferable to external patches which have been used to prevent LV dilation. Furthermore, fibrin glue adheres to various substrates including collagen and cell surface receptors through covalent bonds, hydrogen and other electrostatic bonds, and mechanical interlocking. Therefore, it may prevent myocyte slippage and subsequent LV expansion by binding to the neighboring normal myocardium. Fibrin may also preserve LV function by increasing blood flow to the ischemic tissue. Similar to when delivered in an acute MI, fibrin glue also increased neovasculature formation compared to injection of BSA in our chronic MI model. Natively, fibrin is highly involved in wound healing and acts as the body's natural matrix for neovasculature formation.

While fibrin glue alone preserved cardiac function and LV geometry, the combination of skeletal myoblasts and fibrin glue significantly increased cardiac function and significantly decreased LV expansion compared to BSA, fibrin glue alone, and myoblasts in BSA. In addition to the favorable effects of fibrin alone, myoblasts in fibrin glue may have added benefit by increasing the myoblast density in the infarct area. As when injected into an acute MI, fibrin glue improved cell survival compared to injection in BSA in the chronic MI model. Fibrin glue is believed to increase cell survival in one regard by acting as a temporary extracellular matrix for the transplanted myoblasts. Instead of being an injected carrier that remains something completely liquid post-injection in the tissues, such as saline or BSA, fibrin glue instead solidifies inside the myocardium, giving the cells a temporary semi-rigid scaffold. Fibrin glue also contains RGD motifs and binds to cell receptors, predominately integrins, thus giving the cells a matrix to attach to. Fibrin glue may also increase cell survival by inducing neovasculature formation in ischemic myocardium. An increase in blood supply would provide a less ischemic region for the cells to thrive.

Results according to the present Example further confirm that there were no significant immune reactions in the myocardium related to fibrin glue injections. Fibrin glue is observed to be generally biocompatible, non-toxic, and not generally observed to induce inflammation, foreign body reactions, tissue necrosis or extensive fibrosis. Another benefit of this injectable scaffold is that it is an already FDA approved material, which is routinely used as a surgical adhesive and sealant. Since it remains liquid before combination of its two components, it could also be delivered via catheter, thus requiring only a minimally invasive procedure in humans.

Experiments according to prior Examples confirmed that fibrin glue alone, and myoblasts in fibrin glue, prevent a deterioration of cardiac function when delivered to patients one week after MI. The results according to the experiments of the present Example indicate that skeletal myoblasts delivered in an injectable fibrin scaffold improve cardiac function and decrease LV expansion when delivered five weeks following a MI. Accordingly, delivery of cells in a fibrin scaffold provide a beneficial treatment modality for patients who suffer from a MI, whether delivered soon after the MI or several weeks following it.

Further information related to the methods, materials, or analysis of results according to one or more of the Examples described above, or otherwise providing general background information for further understanding of the embodiments, is variously disclosed in the following references:
1. Mann, D L, “Mechanisms and models in heart failure: A combinatorial approach.” Circulation. 1999; 100:999-1008.
2. Ghostine, S et al., “Long-term efficacy of myoblast transplantation on regional structure and function after myocardial infarction.” Circulation. 2002; 106:I131-I136.
3. Gojo, S et al., “Transplantation of genetically marked cardiac muscle cells. J Thorac Cardiovasc Surg. 1997; 113:10-8.
4. Jain, M et al., “Cell therapy attenuates deleterious ventricular remodeling and improves cardiac performance after myocardial infarction.” Circulation. 2001; 103:1920-7.
5. Li, R K et al., “Cardiomyocyte transplantation improves heart function.” Ann Thorac Surg. 1996; 62:654-60; discussion 660-1.
6. Muller-Ehmsen, J et al., “Survival and development of neonatal rat cardiomyocytes transplanted into adult myocardium. J Mol Cell Cardiol. 2002; 34:107-16.
7. Orlic, D et al., “Bone marrow cells regenerate infarcted myocardium.” Nature. 2001; 410:701-5.
8. Reinecke, H, et al., “Survival, integration, and differentiation of cardiomyocyte grafts: a study in normal and injured rat hearts.” Circulation. 1999; 100:193-202.
9. Reinecke, H, et al., “Transmural replacement of myocardium after skeletal myoblast grafting into the heart. Too much of a good thing? Cardiovasc Pathol. 2000; 9:337-44.
10. Sakai, T et al., “Fetal cell transplantation: a comparison of three cell types.” J Thorac Cardiovasc Surg. 1999; 118:715-24.
11. Scorsin, M et al., “Does transplantation of cardiomyocytes improve function of infarcted myocardium?” Circulation. 1997; 96:II-188-93.
12. Zhang, M et al., “Cardiomyocyte grafting for cardiac repair: graft cell death and anti-death strategies.” J Mol Cell Cardiol. 2001; 33:907-21.
13. Reinecke, H et al., “Taking the death toll after cardiomyocyte grafting: a reminder of the importance of quantitative biology.” J Mol Cell Cardiol. 2002; 34:251-3.
14. Wolfe, C L et al., “Assessment of myocardial salvage after ischemia and reperfusion using magnetic resonance imaging and spectroscopy.” Circulation. 1989; 80:969-82.
15. Zhu, B et al., “Comparative effects of pretreatment with captopril and losartan on cardiovascular protection in a rat model of ischemia-reperfusion. J Am Coll Cardiol. 2000; 35:787-95.
16. Zhu, B et al., “Effects of different durations of pretreatment with losartan on myocardial infarct size, endothelial function, and vascular endothelial growth factor.” J Renin Angiotensin Aldosterone Syst. 2001; 2:129-33.
17. Fishbein, M C et al., “Experimental myocardial infarction in the rat: qualitative and quantitative changes during pathologic evolution.” Am J Pathol. 1978; 90:57-70.
18. Doursout, M F et al., “Measurement of cardiac function in conscious rats.” Ultrasound Med Biol. 2001; 27:195-202.
19. Li, W et al., “Role of MMPs and plasminogen activators in angiogenesis after transmyocardial laser revascularization in dogs.” Am J Physiol Heart Circ Physiol. 2003; 284:H23-30.
20. Havenith, M G et al., “Muscle fiber typing in routinely processed skeletal muscle with monoclonal antibodies.” Histochemistry. 1990; 93:497-9.
21. Kelley, S T et al., “Restraining infarct expansion preserves left ventricular geometry and function after acute anteroapical infarction.” Circulation. 1999; 99:135-42.
22. Thompson, W D et al., “Angiogenic activity of fibrin degradation products is located in fibrin fragment E.” J Pathol. 1992; 168:47-53.
23. Bootle-Wilbraham, C A et al., “Fibrin fragment E stimulates the proliferation, migration and differentiation of human microvascular endothelial cells in vitro.” Angiogenesis. 2001; 4:269-75.
24. Naito, M et al., “Smooth muscle cell outgrowth stimulated by fibrin degradation products. The potential role of fibrin fragment E in restenosis and atherogenesis.” Thromb Res. 2000; 98:165-74.
25. Sahni, A et al., “Binding of basic fibroblast growth factor to fibrinogen and fibrin.” J Biol. Chem. 1998; 273:7554-9.
26. Harrison, P et al., “Platelet alpha-granules.” Blood Rev. 1993; 7:52-62.
27. Horch, R et al., “Cologne Burn Centre experiences with glycerol-preserved allogeneic skin: Part I: Clinical experiences and histological findings (overgraft and sandwich technique).” Burns. 1994; 20 Suppl 1:S23-6.
28. Brennan, M, “Fibrin glue.” Blood Rev. 1991; 5:240-4.
29. Radosevich, M et al., “Fibrin sealant: scientific rationale, production methods, properties, and current clinical use.” Vox Sang. 1997; 72:133-43.

The disclosures of these references just cited above are herein incorporated in their entirety by reference thereto.

Notwithstanding the foregoing description of the various embodiments and further referencing the Examples, and despite what specific mechanisms are in particular involved, it is to be appreciated that the various compound preparations, systems, and methods herein disclosed are nevertheless clearly shown to provide the intended results in treating certain cardiac conditions.

Although the description above contains many details, these should not be construed as limiting the scope of the invention but as merely providing illustrations of some of the presently preferred embodiments of this invention. Moreover, the various aspects, modes, embodiments, variations, or features herein described are considered well suited for further modification and combinations with other known devices and methods for intervening into cardiac structures and providing treatments. For example, the following references are herein incorporated in their entirety by reference thereto: U.S. Pat. No. 6,059,726 to Lee et al.; U.S. Pat. No. 6,129,761 to Hubbell; 6,242,473 to Hellstrand et al.; U.S. Pat. No. 6,312,685 to Fisher et al.; U.S. Pat. No. 6,334,968 to Shapiro et al.; U.S. Pat. No. 6,425,918 to Shapiro et al.; U.S. Pat. No. 6,443,949 to Altman; U.S. Pat. No. 6,502,576 to Lesh; U.S. Pat. No. 6,511,477 to Altman et al.; U.S. Pat. No. 6,533,819 to Urry et al.; U.S. Pat. No. 6,547,787 to Altman et al.; and Published PCT Patent Application No. WO 00/59375 to Sen et al. Within these references are further examples of devices, features, and related methods that may be suitably combined with the description provided herein as would be apparent to one of ordinary skill, and such combinations are considered further aspects of the present invention.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A system for treating a cardiac condition in a patient, comprising:
a volume of living cells; and
a volume of an injectable polymer agent;
wherein the volume of living cells and volume of injectable polymer agent are provided in combination as an injectable scaffolding agent that is characterized as being injectable into a cardiac structure and adapted to provide a therapeutic scaffolding within the cardiac structure.

2. The system of claim 1, wherein the injectable scaffolding agent comprises two precursor agents that are adapted to be combined in-situ.

3. The system of claim 1, wherein the injectable polymer agent comprises a fibrin glue agent.

4. The system of claim 3, wherein the fibrin glue agent comprises fibrinogen and thrombin as two separate precursor material agents.

5. The system of claim 4, wherein the fibrinogen and thrombin are adapted to be injected into the cardiac structure separately such that they form a fibrin glue mixture that polymerizes at least in part within the cardiac structure.

6. The system of claim 4, wherein the fibrinogen and thrombin are adapted to be injected into the cardiac structure in combination as an injectable mixture.

7. The system of claim 4, wherein:
the volume of living cells is combined with the thrombin as an injectable mixture; and
the injectable mixture and the fibrinogen are adapted to be combined as the injectable scaffolding agent.

8. The system of claim 4, wherein:
the volume of living cells is combined with the fibrinogen as an injectable mixture; and
the injectable mixture and the thrombin are adapted to be combined as the injectable scaffolding agent.

9. The system of claim 3, wherein the fibrin glue agent and living cells are adapted to be injected into the cardiac structure separately such that they mix within the cardiac structure to form the therapeutic scaffolding.

10. The system of claim 3, wherein the fibrin glue agent and living cells are adapted to be injected into the cardiac structure combined as an injectable mixture.

11. The system of claim 1, wherein the injectable polymer agent comprises an angiogenic agent.

12. The system of claim 11, wherein the therapeutic scaffolding is adapted to induce therapeutic angiogenesis within the cardiac structure.

13. The system of claim 11, wherein the injectable polymer agent comprises a bioactive fragment E within the cardiac structure.

14. The system of claim 1, wherein the injectable polymer agent is adapted to induce deposition of autologous cells of the patient within the cardiac structure.

15. The system of claim 1, wherein the injectable polymer agent is adapted to enhance retention of the living cells within the therapeutic scaffolding within the cardiac structure.

16. The system of claim 1, wherein the injectable polymer agent comprises a bioactive RDG binding site within the cardiac structure.

17. The system of claim 1, wherein the volume of living cells comprises myoblasts.

18. The system of claim 1, wherein the volume of living cells comprises fibroblasts.

19. The system of claim 1, wherein the volume of living cells comprises stem cells.

20. The system of claim 1, wherein the living cells are genetically modified to express connexin-43.

21. The system of claim 1, wherein the living cells are autologous cells of the patient.

22. The system of claim 1, further comprising:
a cardiac structure injector;
wherein the volume of living cells is coupled to the cardiac structure injector;
wherein the volume of injectable polymer agent is coupled to the cardiac structure injector;
wherein the cardiac structure injector is adapted to inject the volume of living cells and the volume of injectable polymer agent into the cardiac structure in combination as the injectable scaffolding agent in a manner adapted to form the therapeutic scaffolding within the cardiac structure.

23. The system of claim 22, wherein the cardiac structure injector comprises:
an elongate body with a proximal end portion and a distal end portion that is adapted to be delivered to a location associated with the cardiac structure within the patient at least in part by manipulating the proximal end portion externally of the patient; and
a needle injection assembly with at least one injection needle that is extendable from the distal end portion at the location to penetrate the cardiac structure; and
wherein the needle injection assembly is adapted to inject the injectable scaffolding agent into the cardiac structure in a manner that forms the therapeutic scaffolding.

24. The system of claim 23, wherein:
the needle injection assembly comprises a plurality of said injection needles; and
the plurality of the injection needles are adapted to inject the injectable scaffolding agent over a region associated with a damaged portion of the cardiac structure.

25. The system of claim 24, wherein:
at least one electrode adapted to be located along one of the injection needles within the cardiac structure; and
the at least one electrode is coupled to a conductor which is further coupled to a proximal electrical coupler located along the proximal end portion of the elongate body.

26. The system of claim 25, wherein the at least one electrode comprises a mapping electrode.

27. The system of claim 26, further comprising a cardiac conduction mapping system that is adapted to couple to the proximal electrical coupler.

28. The system of claim 26, wherein the mapping electrode is adapted to cooperate with the respective injection needle so as to locate the injection of the injectable scaffolding agent to substantially correspond with the damaged region of the cardiac structure.

29. The system of claim 28, further comprising:
a plurality of said mapping electrodes;
wherein each of the mapping electrodes is adapted to cooperate with a unique one of the plurality of injection needles such that the plurality of injection needles are positionable such that the region corresponding to the injected scaffolding agent substantially corresponds with the damaged portion of the cardiac structure.

30. The system of claim 25, wherein the electrode comprises a cardiac stimulation electrode.

31. The system of claim 30, further comprising a cardiac stimulation assembly with a cardiac stimulation energy source that is adapted to couple to the proximal electrical coupler and to energize the electrode so as to provide cardiac stimulation threshold energy to the cardiac structure.

32. The system of claim 24, further comprising:
an anchor;
wherein the anchor is adapted to secure the needle injection assembly at a desired location along the heart such that the plurality of injection needles may be extended into the cardiac structure.

33. The system of claim 32, wherein:
the plurality of injection needles are extendable from the distal end portion of the elongate body along a circumferential pattern; and
the anchor is located substantially centrally of the circumferential pattern.

34. The system of claim 32, wherein the anchor comprises a screw.

35. The system of claim 32, wherein:
the anchor comprises an electrode; and
the electrode is coupled to a conductor that is further coupled to a proximal electrical coupler located along the proximal end portion.

36. The system of claim 23, wherein the needle injection assembly comprises:
a mixing chamber coupled to both the sources of living cells and injectable polymer agent; and
an injection lumen extendable from the mixing chamber to an injection port located along the needle;
wherein the mixing chamber is adapted to mix the injectable scaffolding agent as a single mixture; and
wherein the injection lumen is adapted to deliver the single mixture to the tissue via the injection port.

37. The system of claim 36, wherein the needle injection assembly further comprises:
first and second delivery lumens;
wherein the source of living cells and source of injectable polymer agent are combined in a manner which forms first and second precursor agents;
wherein the first delivery lumen is coupled to the first precursor agent;
wherein the second delivery lumen is coupled to the second precursor agent; and
wherein the first and second delivery lumens are both coupled to the mixing chamber such that the first and second precursor materials are adapted to be delivered to and mixed within the mixing chamber to form a single injectable mixture.

38. The system of claim 22, wherein the cardiac structure injector comprises an endocardial cardiac structure injection catheter.

39. The system of claim 22, wherein the cardiac structure injector comprises an epicardial cardiac tissue injection catheter.

40. The system of claim 22, wherein the cardiac structure injector comprises a transvascular cardiac tissue injection catheter.

41. The system of claim 22, wherein the cardiac structure injector comprises a guidewire tracking member.

42. The system of claim 41, further comprising a guidewire.

43. The system of claim 22, wherein the cardiac structure injector is deflectable in-situ.

44. The system of claim 43, further comprising a deflection stylet.

45. The system of claim 23, wherein:
the cardiac structure injector comprises an expandable member;
the needle injection assembly cooperates with the expandable member so as to extend the injection needle into the cardiac structure.

46. The system of claim 45, wherein the expandable member comprises an inflatable balloon.

47. The system of claim 1, further comprising:
a kit adapted to combine the volume of living cells and volume of injectable polymer agent in a manner so as to form the injectable scaffolding agent.

48. The system of claim 1, wherein the injectable scaffolding agent is adapted to provide sufficient therapeutic mechanical scaffolding to a ventricular wall so as to prevent substantial progression of left ventricular dysfunction.

49. The system of claim 1, wherein the injectable scaffolding agent is adapted to provide sufficient therapeutic mechanical scaffolding to a ventricular wall so as to prevent progression of cardiomyopathy.

50. The system of claim 1, wherein the injectable scaffolding agent is adapted to provide sufficient therapeutic scaffolding to enhance cardiac function within a region of damaged cardiac tissue.

51. The system of claim 50, wherein the injectable scaffolding agent is adapted to provide sufficient therapeutic scaffolding to enhance cardiac function within a cardiac structure that comprises an infarct.

52. A system for treating a medical condition in a heart of a living being, comprising:
a first injectable composition of material that includes living cells or genetic material; and
a second injectable composition of material that is adapted to enhance retention of the living material in cardiac tissue.

53. The system of claim 52, wherein the first injectable composition of material comprises an autologous cell culture from the living being.

54. The system of claim 52, wherein the first injectable composition of material comprises myoblasts, fibroblasts, skeletal cells, or viruses.

55. The system of claim 52, wherein the second injectable composition of material comprises an injectable polymer.

56. The system of claim 52, wherein the second injectable composition of material comprises a fibrin glue agent.

57. A system for treating a cardiac condition associated with a heart of a patient, comprising:
a cardiac structure injection assembly; and
means associated with the cardiac structure injection assembly for providing a therapeutic scaffolding within a cardiac structure associated with the heart.

58. A system for treating a cardiac condition associated with a heart in a patient, comprising:
a cardiac structure injection assembly;
a volume of living cells coupled to the cardiac tissue injection assembly;
wherein the cardiac structure injection assembly is adapted to inject the volume of living cells into a cardiac structure associated with the heart; and
means coupled to the cardiac structure injection assembly for enhancing the retention of the living cells injected into the cardiac structure.

59. A system for treating a cardiac condition associated with a heart in a patient, comprising:
a volume of injectable polymer agent; and
means for treating the cardiac condition with the volume of injectable polymer agent.

60. A system for repairing a tissue structure in a heart of a patient, comprising:
a first injectable composition of material that includes living cells or genetic material and that is adapted to be injected into the tissue structure; and
means for enhancing retention of the first injectable composition of material in the tissue structure.

61. A system for increasing the size of a chamber wall in a heart of a patient, comprising:
a delivery system; and
a composition of material that is adapted to be delivered into the chamber wall by the delivery system and that comprises means for increasing the size of the chamber wall.

62. The system of claim 61, wherein the means comprises an injectable polymer agent.

63. The system of claim 61, wherein the means comprises an injectable fibrin glue agent.

64-92. (canceled)
